(19) United States
(12) Patent Application Publication
(10) Pub. No.: US 2023/0381190 A1
(43) Pub. Date: Nov. 30, 2023

(54) Methods and Treatment for Complex Lymphatic Malformations

(71) Applicant: Avalo Therapeutics, Inc., Rockville, MD (US)

(72) Inventor: Garry A. Neil, Havertown, PA (US)

(73) Assignee: Avalo Therapeutics, Inc., Rockville, MD (US)

(21) Appl. No.: 18/323,488

(22) Filed: May 25, 2023

Related U.S. Application Data
(60) Provisional application No. 63/346,589, filed on May 27, 2022.

(51) Int. Cl. A61K 31/53; A61P 7/00

(52) U.S. Cl. CPC A61K 31/53; A61P 7/00

(57) ABSTRACT

The present disclosure relates to methods of treating subjects with complex lymphatic malformations, comprising administering to a human subject diagnosed with a complex lymphatic malformation an effective amount of trans-4-[4-Amino-5-(7-methoxyl-1H-indol-2-yl)-imidazo[5,1-f][1,2,4]triazine-7-yl]-cyclohexanecarboxylic acid, or a pharmaceutically acceptable salt thereof at certain dosages.

### CROSS-REFERENCE TO RELATED APPLICATIONS

This application claims the benefit of priority to U.S. Provisional Application No. 63/346,589, filed May 27, 2022, the entire contents of which are incorporated herein by reference for all purposes.

### FIELD OF THE INVENTION

The present disclosure relates to methods of treating subjects with complex lymphatic malformations by administering trans-4-[4-Amino-5-(7-methoxyl-1H-indol-2-yl)-imidazo[5,1-f][1,2,4]triazine-7-yl]-cyclohexanecarboxylic acid, or pharmaceutically acceptable salts thereof.

### BACKGROUND OF THE INVENTION

Lymphatic malformations are a group of rare, serious, congenital diseases with variable clinicopathological presentations. (Ozeki M, Fukao T. Generalized Lymphatic Anomaly and Gorham-Stout Disease: Overview and Recent Insights. Adv Wound Care (New Rochelle). 2019; 8(6):230-245). Lymphatic malformations are localized lesions that consist of dilated lymphatic channels filled with lymph but disconnected from the normal lymphatic system. Lymphatic malformations are congenital and may become infected (Brouillard P, Boon L, Vikkula M. Genetics of lymphatic anomalies. J Clin Invest. 2014; 124(3):898-904). Many patients have common cystic lymphatic malformations, macrocystic, microcystic and mixed (IS SVA Classification of Vascular Anomalies. 2018. International Society for the Study of Vascular Anomalies, Available at “issva.org/classification” Accessed June 2018). These are the most common lymphatic anomalies, with most occurring in infants (Ozeki and Fukao, Adv Wound Care (2019)). These lesions are solitary, cystic, soft-tissue masses that are mostly found in neck, mediastinum, and retroperitoneum. Many patients across this spectrum of diseases have chronic, intractable symptoms and severe disability without approved therapies (Trenor C C 3rd, Chaudry G. Complex lymphatic anomalies. Semin Pediatr Surg, 2014; 23(4):186-190). There is a mortality of approximately 20% at 7 years (Ozeki M, Fujino A, Matsuoka K, Nosaka S, Kuroda T, Fukao T. Clinical features and prognosis of generalized lymphatic anomaly, kaposiform lymphangiomatosis, and Gorham-Stout disease. Pediatr Blood Cancer 2016; 63:832-838).

The current approach to treatment of lymphatic malformations is “tailored” to clinical diagnosis, complications, and the size and location of the lesion(s). Surgery and interventional therapy are performed in patients with lymphatic anomalies, as required. No drug treatments are currently approved for treatment of these conditions. Thus, there is a need for improved treatments for complex lymphatic malformations.

Aggressive treatment, including medical and surgical treatment, is necessary for cases of more than moderate severity. Bisphosphonates are frequently used for the treatment of patients with osteolysis (Kuriyama D K, McElligott S C, Glaser D W, Thompson K S. Treatment of Gorham-Stout disease with zoledronic acid and interferon-alpha: a case report and literature review. J Pediatr Hematol Oncol 2010; 32:579-584). Interferons have also been used to inhibit the proliferation of blood and lymphatic vessels, although they are frequently not effective and not well tolerated (Ozeki M, Funato M, Kanda K, et al. Clinical improvement of diffuse lymphangiomatosis with pegylated interferon alfa-2b therapy: case report and review of the literature. Pediatr Hematol Oncol 2007; 24:513-524). Other pharmaceuticals including the anti-vascular endothelial growth factor (VEGF)-A antibody bevacizumab (Grunewald T G, Damke L, Maschan M, et al. First report of effective and feasible treatment of multifocal lymphangiomatosis (Gorham-Stout) with bevacizumab in a child. Ann Oncol. 2010:21(8):1733-1734), propranolol (Ozeki M, Fukao T, Kondo N. Propranolol for intractable diffuse lymphangiomatosis. N Engl J Med 2011; 364:1380-1382), steroids, vitamin D, and calcitonin have also been used with limited success (Ozeki and Fukao, Adv Wound Care (2019)).

Many patients with lymphatic malformations have mutations in the phosphatidylinositol 3-kinase (PI3K)/AKT signaling pathway (Brouillard et al, J Clin Invest (2014)). This pathway is critical to cell growth and survival and has been shown to govern normal vascular development and angiogenesis (Adams D M, Trenor C C 3rd, Hammill A M, et al. Efficacy and Safety of Sirolimus in the Treatment of Complicated Vascular Anomalies. Pediatrics. 2016; 137(2):e20153257). Enhanced mammalian target of rapamycin (mTOR) signaling increases expression of VEGF, a key regulator of angiogenesis and lymphangiogenesis (Adams et al, Pediatrics (2016), Lee D F, Hung M C. All roads lead to mTOR: integrating inflammation and tumor angiogenesis. Cell Cycle. 2007; 6(24):3011-3014). Disorders that lead to inappropriate activation of the PI3K/AKT/mTOR pathway have been shown to cause association with vascular anomalies (Adams et al, Pediatrics (2016)). The discovery of somatic mutations in the PI3K/mTOR pathway (PIK3CA) and mutations, both somatic and germline, in complementary pathways (TIE2, RASA1, and phosphatase tensin homolog [PTEN]) provide compelling evidence for the critical role of this pathway in the regulation of vascular growth and organization. The mTOR integrates signals from the PI3K/AKT pathway to coordinate proper cell growth and proliferation by regulating ribosomal biogenesis and protein synthesis (Adams et al, Pediatrics (2016)).

AVTX-006 (also previously known as CERC-006) is an orally available small molecule that is a potent inhibitor of the mTOR kinase catalytic activities of both mTOR complex 1 (regulatory-associated protein of mTOR [raptor] complex) (mTORC1) and mTOR complex 2 (rapamycin insensitive component of mTOR [rictor] complex) (mTORC2) in biochemical and cellular assays. Together, the mTOR complexes regulate cell growth, proliferation, and survival. Dual mTOR kinase inhibitors were developed to maximize the pharmacological impact of mTOR blockade in comparison with that observed with sirolimus or rapalogs. An orally bioavailable small molecule dual mTORC 1/C2 ATP-competitive kinase inhibitor would represent a “first-in-class” and might demonstrate superior clinical efficacy compared with rapamycin and its analogs.

AVTX-006 is a synthetic potent inhibitor of the mTOR kinase catalytic activities of both mTORC1 and mTORC2 in biochemical and cellular assays. The 50% inhibitory concentration (IC50) value for AVTX-006 was 4 nM. Immunoprecipitation with anti-Raptor antibodies to evaluate mTORC1 activity in the presence of drug, or anti-Rictor antibodies to assess mTORC2 activity indicated that AVTX-006 is equipotent in the inhibition of both functional complexes. Greater than 100-fold selectivity was observed for mTOR relative to other PI3K-related kinases in biochemical assays. To determine the selectivity profile, AVTX-006 (1 μM) was tested against 101 kinases using Caliper kinase profiling assays. No kinases were inhibited at greater than 50%. These data, fully described in 2 publications (Bhagwat S V, Gokhale P C, Crew A P, Cooke A, Yao Y, Mantis C, et al. Preclinical characterization of OSI 027, a potent and selective inhibitor of mTORC1 and mTORC2: distinct from rapamycin. Mol Cancer Ther. Epub 2011 Jun. 14; Falcon B L, Barr S, Gokhale P C, Chou J, Fogarty J, Depeille P, et al. Reduced VEGF production, angiogenesis, and vascular regrowth contribute to the antitumor properties of dual mTORC1/mTORC2 inhibitors. Cancer Res. 2011; 71(5):1573-83) indicate that AVTX-006 is a potent and selective mTOR kinase (mTORC1/mTORC2) inhibitor. In vitro and in vivo pharmacology studies demonstrated that orally administered AVTX-006 has the potential for antitumor efficacy in a range of human cancers that depend on the mTOR pathway for tumor growth and survival.

AVTX-006 was studied in a multicenter, open-label, dose escalation Phase 1 study in 128 subjects with advanced solid tumors or lymphoma (Study OSI-027-101). Mateo et al (Mateo J, Olmos D, Dumez H, et al. A first in man, dose-finding study of the mTORC1/mTORC2 inhibitor OSI-027 in patients with advanced solid malignancies. Br J Cancer. 2016; 114(8):889-896). This study had 3 treatment schedules: once daily for 3 consecutive days every 7 days (Schedule 1 [S1], also referred to as an intermittent dose schedule), single dose every 7 days or once weekly for 3 weeks (Schedule 2 [S2], also referred to as a weekly dose schedule), and once daily each day (Schedule 3 [S3], also referred to as a continuous dose schedule).

The maximum tolerated dose (MTD) of AVTX-006 was 120 mg for 51 and was not reached for S2 (dose escalation stopped at 240 mg every week due to the number of capsules required). Renal toxicity was dose limiting for S3 at 40 mg once daily, therefor the maximum tolerated dose (MTD) for once daily dosing was 30 mg once daily. The observed dose limiting toxicities (DLTs) were mainly fatigue, renal, and cardiac events. Fatigue and renal toxicity were frequent causes of dose reduction after Cycle 1. The 90 mg (S1), 120 mg (S1) and 240 mg (S2) cohorts were selected for assessing pharmacodynamics (PD) in specific expansion cohorts.

Of the 128 subjects enrolled, 123 subjects received AVTX-006. The most frequently reported treatment-emergent adverse events (TEAEs) (>10% of subjects), regardless of relationship to study drug, were fatigue (65.0%); nausea (60.2%); anorexia (40.7%); vomiting (33.3%); blood creatinine increased (28.5%); constipation (25.2%); diarrhea (22.8%); abdominal pain and dyspnea (17.1% each); headache, cough, and back pain (14.6%); anemia and weight decreased (13.8%), hypokalemia (12.2%); and abdominal pain upper (10.6%). Overall, 92 of 123 subjects (74.8%) reported TEAEs considered drug regimen-related by the investigator. The most frequently reported drug regimen related TEAEs included fatigue (44.7%), nausea (40.7%), increased blood creatinine (26.0%), anorexia (21.1%) and vomiting (18.7%).

Fifty subjects of 123 subjects treated (40.6%) experienced serious adverse events (SAEs). Fifteen subjects who received AVTX-006 (12.2%) had related SAEs, including nausea and fatigue (3 subjects each); blood creatinine increased and vomiting (2 subjects each), vomiting (2 subjects), diarrhea, anorexia, pneumonia, cardiac failure congestive, myocardial infarction, stress cardiomyopathy, supraventricular tachycardia, bone pain, renal failure, cataract subcapsular, and urticaria (1 subject each). Deaths were reported for 48.2% of subjects in S1, 25.6% of subjects in S2 and 14.3% of subjects in S3. None of the deaths were considered drug related.

AVTX-006 was absorbed after oral dosing with peak plasma concentrations (Cₘₐₓ) reached at 4 hours. The time to maximum observed serum concentration (tₘₐₓ) was similar across dose groups, suggesting the rate of absorption of AVTX-006 was independent of dose. In both S1 and S2, the area under the curve (AUC) of AVTX-006 was dose proportional (slope was close to 1). Increases in Cₘₐₓ were apparently proportional to dose in S1 and S3 but not in S2. Conclusions about dose proportionality are made cautiously because of the small subject numbers in each group. The estimated terminal half-life (t_{1/2}) of AVTX-006, approximately 14 hours, was calculated from the 240 mg dose in S2 (weekly dose schedule), a cohort with sufficient samples in the terminal phase to estimate t_{1/2}. The range of t_{1/2} in the other dose groups was 8 to 25 hours. The apparent oral clearance in S2 was relatively low, ranging from 0.6 to 2.0 L/h. There was minimal accumulation of AVTX-006 observed after once daily dosing (the accumulation ratio ranged from 1.1 to 1.5).

On the basis of the PD and tolerability data from OSI-027-101 study, development of AVTX-006 as a single agent in subjects with advanced solid tumors or lymphoma was discontinued.

### SUMMARY

The present disclosure includes, for example, any one or a combination of the following embodiments:
Embodiment 1. A method of treating a complex lymphatic malformation (CLM), comprising administering to a human subject diagnosed with a CLM trans-4-[4-Amino-5-(7-methoxyl-1H-indol-2-yl)-imidazo[5,1-f][1,2,4]triazine-7-yl]-cyclohexanecarboxylic acid, or a pharmaceutically acceptable salt thereof, wherein the trans-4-[4-Amino-5-(7-methoxyl-1H-indol-2-yl)-imidazo[5,1-f][1,2,4]triazine-7-yl]-cyclohexanecarboxylic acid, or pharmaceutically acceptable salt thereof is administered to the subject at a dose of 1 mg/day or 2 mg/day.
Embodiment 2. The method of embodiment 1, wherein the trans-4-[4-Amino-5-(7-methoxyl-1H-indol-2-yl)-imidazo[5,1-f][1,2,4]triazine-7-yl]-cyclohexanecarboxylic acid, or pharmaceutically acceptable salt thereof is administered in a 28-day cycle.
Embodiment 3. The method of embodiment 1 or 2, wherein the dose is 0.5 mg administered twice daily.
Embodiment 4. The method of embodiment 1 or 2, wherein the dose is 1 mg administered twice daily.
Embodiment 5. The method of embodiment 1, wherein the 0.5 mg twice daily dose comprises a first 0.5 mg dose and a second 0.5 mg dose, wherein the second 0.5 mg dose is administered about 12 hours after the first 0.5 mg dose.
Embodiment 6. The method of embodiment 1, wherein the 1 mg twice daily dose comprises a first 1 mg dose and a second 1 mg dose, wherein the second 1 mg dose is administered about 12 hours after the first 1 mg dose.
Embodiment 7. The method of any one of embodiments 1-6, wherein the trans-4-[4-Amino-5-(7-methoxyl-1H-indol-2-yl)-imidazo[5,1-f][1,2,4]triazine-7-yl]-cyclohexanecarboxylic acid, or pharmaceutically acceptable salt thereof is administered as an oral solution.
Embodiment 8. The method of any one of embodiments 1-7, wherein the pharmaceutically acceptable salt is trans-4-[4-Amino-5-(7-methoxyl-1H-indol-2-yl)-imidazo[5,1-f][1,2,4]triazine-7-yl]-cyclohexanecarboxylic acid, tromethamine salt.
Embodiment 9. The method of any one of embodiments 1-8, wherein the complex lymphatic malformation is selected from generalized lymphatic anomaly, Gorham syndrome, kaposiform lymphangiomatosis, microcystic lymphatic malformation, capillary lymphatico/venous malformation, abnormalities of the central conducting lymphatic channels, venous lymphatic malformation, capillary lymphatic arterial venous malformation, and phosphatase tensin homolog (PTEN) hamartoma tumor syndrome/vascular lesions.
Embodiment 10. The method of any one of embodiments 1-9, wherein the complex lymphatic malformation is characterized by abnormal formation of lymphatic vessels and/or tissue overgrowth.
Embodiment 11. The method of any one of embodiments 1-9, wherein the subject has at least one localized lesion.
Embodiment 12. The method of embodiment 12, wherein the lesion involves fluid accumulation in the scalp, face, neck, limbs, abdomen, and/or chest.
Embodiment 13. The method of embodiment 11 or 12, wherein the subject exhibits at least a 20% reduction in the size of the lesion from baseline to end of treatment as assessed using radiologic imaging.
Embodiment 14. The method of embodiment 11 or 12, wherein the subject achieves no evidence of disease as assessed using radiologic imaging.
Embodiment 15. The method of embodiment 13 or 14, wherein the radiologic imaging is selected from magnetic resonance imaging (MRI), computed tomography (CT), and ultrasound.
Embodiment 16. The method of any one of embodiments 1-9, wherein the lymphatic malformation is a cystic lymphatic malformation.
Embodiment 17. The method of embodiment 16, wherein the cystic lymphatic malformation is selected from macrocystic, microcystic, and mixed.
Embodiment 18. The method of any one of embodiments 1-9, wherein the complex lymphatic malformation is classified as moderate to severe based on medical history, imaging, and/or disease-related complications requiring systemic control.
Embodiment 19. The method of embodiment 18, wherein the complication is selected from coagulopathy, chronic pain, recurrent cellulitis, ulceration, visceral and/or bone involvement, and cardiac dysfunction.
Embodiment 20. The method of any one of embodiments 1-18, wherein the subject exhibits a reduction in pain as assessed on a Visual Analogue Scale (VAS).
Embodiment 21. The method of any one of embodiments 1-18, wherein the subject exhibits an improvement in quality-of-life as assessed using 36-Item Short Form Health Survey (SF-36).
Embodiment 22. The method of any one of embodiments 1-18, wherein the subject exhibits an improvement in Karnofsky Performance Status.
Embodiment 23. The method of any one of embodiments 1-22, wherein the subject is not administered a treatment selected from sirolimus, mitogen-activated protein kinase (MEK) inhibitors, PIK3CA inhibitors, or interferon alfa 2b, vascular endothelial growth factor receptor 3 (VEGFR-3) inhibitors, erlotinib or other epidermal growth factor receptor inhibitor, a systemic steroid, other immunosuppressive agents and/or other systemic agents targeting lymphatic malformations at the same time as the trans-4-[4-Amino-5-(7-methoxyl-1H-indol-2-yl)-imidazo[5,1-f][1,2,4]triazine-7-yl]-cyclohexanecarboxylic acid, or pharmaceutically acceptable salt thereof.
Embodiment 24. The method of any one of embodiments 1-22, wherein if the subject has received treatment with sirolimus, mitogen-activated protein kinase (MEK) inhibitors, PIK3CA inhibitors, or interferon alfa 2b, vascular endothelial growth factor receptor 3 (VEGFR-3) inhibitors, and/or other systemic agents targeting lymphatic malformations, the subject has undergone the required 14-day washout period prior to a baseline visit.
Embodiment 25. The method of any one of embodiments 1-22, wherein if the subject has received treatment with a topical agent, other than a topical steroid, targeting lymphatic malformations, the subject has undergone the required 7-day washout period prior to a baseline visit.
Embodiment 26. The method of any one of embodiments 1-22, wherein the subject is administered a topical steroid targeting lymphatic malformations at the same time as the trans-4-[4-Amino-5-(7-methoxyl-1H-indol-2-yl)-imidazo[5,1-f][1,2,4]triazine-7-yl]-cyclohexanecarboxylic acid, or pharmaceutically acceptable salt thereof.
Embodiment 27. The method of any one of embodiments 1-26, wherein the subject does not have a serum creatinine concentration of >1.5 mg/dl.
Embodiment 28. The method of any one of embodiments 1-26, wherein the subject does not have hemoglobin ≤8 g/dl, a peripheral absolute neutrophil count (ANC)≤1,000/μl and/or a platelet count <50,000/μl.
Embodiment 29. The method of any one of embodiments 1-28, wherein the subject does not have total bilirubin (sum of conjugated and unconjugated)>1.5×upper limit of normal (ULN), Aspartate transaminase/Alanine aminotransferase (AST/ALT)≥5×ULN and serum albumin ≤2 g/dL.
Embodiment 30. The method of any one of embodiments 1-28, wherein the subject is a pediatric subject.

### DETAILED DESCRIPTION OF THE INVENTION

The following definitions are provided to facilitate an understanding of the invention. They are not intended to limit the invention in any way.

### Definitions

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e., combinations) of two or more of the compounds. According to the present invention, an “isolated,” or “biologically pure” molecule is a compound that has been removed from its natural milieu. As such, the terms “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition. “Or” is used in the inclusive sense, i.e., equivalent to “and/or,” unless the context requires otherwise.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone). The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

The terms “inhibition” or “inhibit” refer to a decrease or cessation of any event (such as protein ligand binding) or to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. It is not necessary that the inhibition or reduction be complete. For example, in certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

“Sample” or “subject sample” or “biological sample” generally refers to a sample which may be tested for a particular molecule. Samples may include but are not limited to cells, bone marrow, body fluids, including blood, serum, plasma, urine, saliva, stool, tears, pleural fluid and the like.

A “subject” can be mammalian. In any of the embodiments involving a subject, the subject can be human. In any of the embodiments involving a subject, the subject can be a cow, pig, monkey, sheep, dog, cat, fish, or poultry.

A “pediatric” subject herein is a human of less than 18 years of age, whereas an “adult” subject is 18 years or older.

The term “pharmaceutically acceptable composition” may refer to a composition comprising AVTX-006 in formulations with a wide variety of pharmaceutically acceptable carriers.

The term “pharmaceutically acceptable carrier” refers to refers to an ingredient in a pharmaceutical formulation or composition, other than an active ingredient, which is nontoxic to a subject. A pharmaceutically acceptable carrier includes, but is not limited to, a buffer, excipient, stabilizer, and/or preservative.

The term “administering”, “administered” and grammatical variants refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In one embodiment, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

“Treatment” or “treat” refers to both therapeutic treatment and prophylactic or preventative measures. Those in need of treatment include those already with the disorder as well as those prone to have the disorder or those in which the disorder is to be prevented. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The term “effective amount” or “therapeutically effective amount” refers to an amount of a drug effective for treatment of a disease or disorder in a subject, such as to partially or fully relieve one or more symptoms. In some embodiments, an effective amount refers to an amount effective, at dosages and for periods of time necessary, to achieve the desired therapeutic or prophylactic result.

### Complex Lymphatic Malformations (CLMs)

“Complex lymphatic malformations (CLMs)”, as used herein, refers to a disease or disorder characterized by localized lesions that consist of dilated lymphatic channels filled with lymph but disconnected from the normal lymphatic system. Non-limiting examples of complex lymphatic malformations include generalized lymphatic anomaly, Gorham syndrome or Gorham Stout Disease, kaposiform lymphangiomatosis, microcystic lymphatic malformation, capillary lymphatico/venous malformation, abnormalities of the central conducting lymphatic channels, venous lymphatic malformation, capillary lymphatic arterial venous malformation, phosphatase tensin homolog (PTEN) hamartoma tumor syndrome/vascular lesions, and other moderate to severe vascular anomalies with associated lymphatic involvement (as described in the current ISSVA classification, ISSVA General Assembly, 2018).

“Moderate to severe complex lymphatic malformations” are those considered, in the opinion of the investigator, to be moderate to severe based on medical history, imaging, and/or disease-related complications requiring systemic control. Examples of disease-related complications include, but are not limited to, coagulopathy, chronic pain, recurrent cellulitis, ulceration, visceral and/or bone involvement, and cardiac dysfunction.

Clinically, lymphangiomas are classified into several types. These include (1) Simplex, which is made up of capillary sized, thin-walled lymphatic channels. This type usually affects the skin (lymphangioma circumscriptum); (2) Cystic lymphangioma (or cystic hygroma): this may range in size from a few millimeters to several centimeters, seen in a young age, commonly in the neck or the axilla; (3) Cavernosum: this type is made up of dilated lymphatic channels, often 25 with fibrous adventitial coats. This is the type which usually affects organs in the thorax, abdomen, and bones. Each of these lymphangiomas are encompassed in the invention.

Complex lymphatic malformations can be classified by whether they are localized (cystic) or not (generalized). Cystic complex lymphatic malformations may be macrocystic (>1 centimeter [cm] in diameter), microcystic (<1 cm in diameter), or of mixed type (both macro- and micro-cysts present). Macrocystic CLM is most likely to occur on the head or neck, while microcystic CLM is often associated with deep infiltration into the body (García-Montero, et al. Pediatrics 2017; 139(5), e20162105. doi:10.1542/peds.2016-2105). Some complex lymphatic malformations are large, diffuse masses capable of infiltrating adjacent tissues and organs (Wiegand et al. Lymphatic Research and Biology 2018; 16(4), 330-339. doi:10.1089/lrb.2017.0062). These diffuse LMs are referred to as generalized lymphatic anomalies (GLAs) (Wu et al. PLoS One 2015 10(2), e0117352. doi:10.1371/journal.pone.0117352). Some GLA patients have LM in many areas of the body, for example, bone involvement, osteolysis in the vertebrae, cystic lesions in the spleen. Treatment of CLM has three main goals: reducing restrictions on patient functionality, controlling symptoms, and avoiding disfiguration.

In some embodiments, diagnosis of a complex lymphatic malformation is made solely based on clinical presentation, scanning results, and/or family history. In some embodiments, diagnosis of a complex lymphatic malformation is made without testing for genetic sequence information. In some embodiments, diagnosis of a complex lymphatic malformation is made based on clinical presentation together with genetic sequence information.

### AVTX-006

As used herein, “AVTX-006”, also known as CERC-006, ASP7486 and OSI-027, means a compound having the chemical structure:

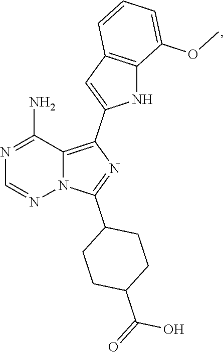 [SMILES: COc1cccc2cc(-c3nc(C4CCC(C(=O)O)CC4)n4ncnc(N)c34)[nH]c12]

and pharmaceutically acceptable salts thereof. AVTX-006 is disclosed in U.S. Pat. No. 7,700,594 B2, which is incorporated herein by reference in its entirety.

In some embodiments, AVTX-006 is 4-[4-Amino-5-(7-methoxyl-1H-indol-2-yl)-imidazo[5,1-f][1,2,4]triazine-7-yl]-cyclohexanecarboxylic acid, or a pharmaceutically acceptable salt thereof. In some embodiments, AVTX-006 refers to the trans isomer, the cis isomer, or a mixture of the trans and cis isomers of 4-[4-Amino-5-(7-methoxyl-1H-indol-2-yl)-imidazo[5,1-f][1,2,4]triazine-7-yl]-cyclohexanecarboxylic acid.

In some embodiments, AVTX-006 is trans-4-[4-Amino-5-(7-methoxyl-1H-indol-2-yl)-imidazo[5,1-f][1,2,4]triazine-7-yl]-cyclohexanecarboxylic acid, or a pharmaceutically acceptable salt thereof. In some embodiments, AVTX-006 is 4-[4-Amino-5-(7-methoxyl-1H-indol-2-yl)-imidazo[5,1-f][1,2,4]triazine-7-yl]-cyclohexanecarboxylic acid, tromethamine salt. In some embodiments, AVTX-006 is trans-4-[4-Amino-5-(7-methoxyl-1H-indol-2-yl)-imidazo[5,1-f][1,2,4]triazine-7-yl]-cyclohexanecarboxylic acid, tromethamine salt.

In some embodiments, AVTX-006 is:

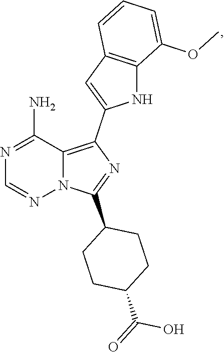 [SMILES: COc1cccc2cc(-c3nc([C@H]4CC[C@H](C(=O)O)CC4)n4ncnc(N)c34)[nH]c12]

or a pharmaceutically acceptable salt thereof.

In some embodiments, AVTX-006 is:

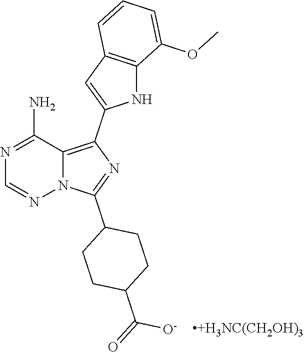

In some embodiments, AVTX-006 is:

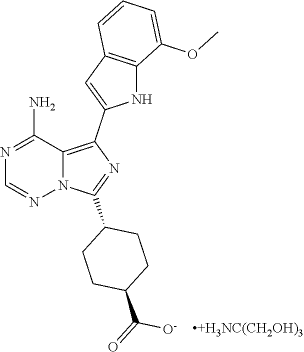

### Pharmaceutical Formulations

AVTX-006 may be administered in the form of a pharmaceutically acceptable composition. In various embodiments, compositions comprising AVTX-006 are provided in formulations with a wide variety of pharmaceutically acceptable carriers (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

In various embodiments, compositions comprising AVTX-006 may be formulated for injection or infusion, by dissolving, suspending, or emulsifying them in an aqueous or nonaqueous solvent, such as vegetable or other oils, synthetic aliphatic acid glycerides, esters of higher aliphatic acids, or propylene glycol; and if desired, with conventional additives such as solubilizers, isotonic agents, suspending agents, emulsifying agents, stabilizers and preservatives. In various embodiments, the compositions may be formulated for inhalation, for example, using pressurized acceptable propellants such as dichlorodifluoromethane, propane, nitrogen, and the like. The compositions may also be formulated, in various embodiments, into sustained release microcapsules, such as with biodegradable or non-biodegradable polymers. A non-limiting exemplary biodegradable formulation includes poly lactic acid-glycolic acid polymer. A non-limiting exemplary non-biodegradable formulation includes a polyglycerin fatty acid ester. Certain methods of making such formulations are described, for example, in EP 1 125 584 A1.

In some embodiments, AVTX-006 is administered as a hard gelatine capsules containing 5 or 20 mg of AVTX-006 plus microcrystalline cellulose, hydroxypropyl cellulose, croscarmellose sodium, and magnesium stearate as excipients.

In some embodiments, AVTX-006 is administered as an oral solution.

Pharmaceutical packs and kits comprising one or more containers, each containing one or more doses of an AVTX-006 are also provided. In some embodiments, a unit dosage is provided wherein the unit dosage contains a predetermined amount of a composition comprising AVTX-006, with or without one or more additional agents. In some embodiments, such a unit dosage is supplied in single-use prefilled syringe for injection. In various embodiments, the composition contained in the unit dosage may comprise saline, sucrose, or the like; a buffer, such as phosphate, or the like; and/or be formulated within a stable and effective pH range. Alternatively, in some embodiments, the composition may be provided as a lyophilized powder that may be reconstituted upon addition of an appropriate liquid, for example, sterile water. In some embodiments, the composition comprises one or more substances that inhibit protein aggregation, including, but not limited to, sucrose and arginine. In some embodiments, a composition of the invention comprises heparin and/or a proteoglycan.

Treatment with AVTX-006

In some embodiments, a method of treating subjects having a complex lymphatic malformation is provided comprising administering an effective amount of trans-4-[4-Amino-5-(7-methoxyl-1H-indol-2-yl)-imidazo[5,1-f][1,2,4]triazine-7-yl]-cyclohexanecarboxylic acid, or pharmaceutically acceptable salt thereof to a human subject diagnosed with a complex lymphatic malformation.

In some embodiments the effective amount of trans-4-[4-Amino-5-(7-methoxyl-1H-indol-2-yl)-imidazo[5,1-f][1,2,4]triazine-7-yl]-cyclohexanecarboxylic acid, or pharmaceutically acceptable salt thereof is a dose of 1 mg/day or 2 mg/day. In some embodiments the effective amount of trans-4-[4-Amino-5-(7-methoxyl-1H-indol-2-yl)-imidazo[5,1-f][1,2,4]triazine-7-yl]-cyclohexanecarboxylic acid, or pharmaceutically acceptable salt thereof is a dose of 1 mg/day. In some embodiments the effective amount of trans-4-[4-Amino-5-(7-methoxyl-1H-indol-2-yl)-imidazo[5,1-f][1,2,4]triazine-7-yl]-cyclohexanecarboxylic acid, or pharmaceutically acceptable salt thereof is a dose of 2 mg/day.

In some embodiments, the 1 mg/day dose is 0.5 mg administered twice daily. In some embodiments, the 2 mg/day dose is 1 mg administered twice daily. In some embodiments, the twice daily dose is administered about 8 hours apart, about 9 hours apart, about 10 hours apart, about 11 hours apart, about 12 hours apart, about 13 hours apart, about 14 hours apart, about 15 hours apart, or about 16 hours apart. In some embodiments, the twice daily dose is administered about 12 hours apart.

In some embodiments, the subject has a verified diagnosis of a complex lymphatic malformation selected from generalized lymphatic anomaly, Gorham syndrome, kaposiform lymphangiomatosis, microcystic lymphatic malformation, capillary lymphatico/venous malformation, abnormalities of the central conducting lymphatic channels, venous lymphatic malformation, capillary lymphatic arterial venous malformation, and phosphatase tensin homolog (PTEN) hamartoma tumor syndrome/vascular lesions.

In some embodiments, the complex lymphatic malformation is characterized by abnormal formation of lymphatic vessels and/or tissue overgrowth. In some embodiments, the subject has at least one localized lesion. In some embodiments the subject has one localized lesion. In some embodiments the subject has two localized lesions. In some embodiments the subject has three localized lesions. In some embodiments, the lesion involves fluid accumulation in the scalp, face, neck, limbs, abdomen, and/or chest. In some embodiments, the lesion occurs on the head or neck.

In some embodiments, the subject exhibits at least a 20% reduction in the size of the lesion from baseline after treatment with trans-4-[4-Amino-5-(7-methoxyl-1H-indol-2-yl)-imidazo[5,1-f][1,2,4]triazine-7-yl]-cyclohexanecarboxylic acid, or pharmaceutically acceptable salt thereof as assessed using radiologic imaging. In some embodiments, the subject exhibits at least a 5%, at least a 10%, at least a 15%, at least a 20%, at least a 25%, at least a 30%, at least a 35%, at least a 40%, at least a 45%, or at least a 50% reduction in the size of the lesion from baseline after treatment with trans-4-[4-Amino-5-(7-methoxyl-1H-indol-2-yl)-imidazo[5,1-f][1,2,4]triazine-7-yl]-cyclohexanecarboxylic acid, or pharmaceutically acceptable salt thereof as assessed using radiologic imaging. In some embodiments, the subject achieves no evidence of disease as assessed using radiologic imaging. In some embodiments the radiologic imaging is selected from magnetic resonance imaging (MRI), computed tomography (CT), and ultrasound. In some embodiments the radiologic imaging is magnetic resonance imaging (MRI).

In some embodiments, the complex lymphatic malformation is a cystic lymphatic malformation. In some embodiments, the cystic lymphatic malformation is selected from macrocystic, microcystic, and mixed.

In some embodiments, the complex lymphatic malformation is classified as moderate to severe based on medical history, imaging, and/or disease-related complications requiring systemic control. In some embodiments, the complication is selected from coagulopathy, chronic pain, recurrent cellulitis, ulceration, visceral and/or bone involvement, and cardiac dysfunction. In some embodiments, the subject exhibits a reduction of the signs and symptoms of disease-related complications.

In some embodiments, the subject exhibits a reduction in pain as assessed on a Visual Analogue Scale (VAS). In some embodiments, the subject exhibits an improvement in quality-of-life as assessed using 36-Item Short Form Health Survey (SF-36). In some embodiments, the subject exhibits an improvement in Karnofsky Performance Status. In some embodiments, the subject exhibits at least a 10%, at least a 20%, at least a 30%, at least a 40%, or at least a 50% improvement in the Karnofsky Performance Status from baseline after treatment with trans-4-[4-Amino-5-(7-methoxyl-1H-indol-2-yl)-imidazo[5,1-f][1,2,4]triazine-7-yl]-cyclohexanecarboxylic acid, or pharmaceutically acceptable salt thereof as assessed by a physician and/or patient.

In some embodiments, the subject is not administered a treatment selected from sirolimus, mitogen-activated protein kinase (MEK) inhibitors, PIK3CA inhibitors, or interferon alfa 2b, vascular endothelial growth factor receptor 3 (VEGFR-3) inhibitors, erlotinib or other epidermal growth factor receptor inhibitor, a systemic steroid, other immunosuppressive agents and/or other systemic agents targeting lymphatic malformations at the same time as the trans-4-[4-Amino-5-(7-methoxyl-1H-indol-2-yl)-imidazo[5,1-f][1,2,4]triazine-7-yl]-cyclohexanecarboxylic acid, or pharmaceutically acceptable salt thereof. In some embodiments, if the subject has received treatment with sirolimus, mitogen-activated protein kinase (MEK) inhibitors, PIK3CA inhibitors, or interferon alfa 2b, vascular endothelial growth factor receptor 3 (VEGFR-3) inhibitors, and/or other systemic agents targeting lymphatic malformations, the subject has undergone the required 14-day washout period prior to a baseline visit. In some embodiments, if the subject has received treatment with a topical agent, other than a topical steroid, targeting lymphatic malformations, the subject has undergone the required 7-day washout period prior to a baseline visit.

In some embodiments, the subject is administered a topical steroid targeting lymphatic malformations at the same time as the trans-4-[4-Amino-5-(7-methoxyl-1H-indol-2-yl)-imidazo[5,1-f][1,2,4]triazine-7-yl]-cyclohexanecarboxylic acid, or pharmaceutically acceptable salt thereof.

In some embodiments, the subject does not have a serum creatinine concentration of >1.5 mg/dl. In some embodiments, the subject does not have hemoglobin ≤8 g/dl, a peripheral absolute neutrophil count (ANC)≤1,000/μl and/or a platelet count <50,000/μl. In some embodiments, the subject does not have total bilirubin (sum of conjugated and unconjugated)>1.5×upper limit of normal (ULN), Aspartate transaminase/Alanine aminotransferase (AST/ALT)≥5×ULN and serum albumin ≤2 g/dL.

In some embodiments, the subject is an adult subject. In some embodiments, the subject is a pediatric subject.

### Kits And Articles of Manufacture

Any of the aforementioned methods can be implemented via kits for the treatment of a complex lymphatic malformation. The kit may contain 4-[4-Amino-5-(7-methoxyl-1H-indol-2-yl)-imidazo[5,1-f][1,2,4]triazine-7-yl]-cyclohexanecarboxylic acid, or a pharmaceutically acceptable salt thereof, a pharmaceutically acceptable carrier, a physiologically acceptable carrier, instructions for use, a container, a vessel for administration, or any combination thereof.

### EXAMPLES

The following examples are provided to illustrate certain disclosed embodiments and are not to be construed as limiting the scope of this disclosure in any way. In the Examples discussed below, “AVTX-006” refers to trans-4-[4-Amino-5-(7-methoxyl-1H-indol-2-yl)-imidazo[5,1-f][1,2,4]triazine-7-yl]-cyclohexanecarboxylic acid, tromethamine salt.

### Example 1—A Phase 1b, Open-Label, Pharmacokinetic, Pharmacodynamic, Safety and Tolerability Study of AVTX-006 in Adults (Aged 18-31 Years) with Complex Lymphatic Malformations

### Example 1.1—Study Objectives and Endpoints

The primary objective of the study is to evaluate the safety and tolerability of AVTX-006 in adults (aged 18-31 years) with active, moderate to severe complex lymphatic malformations.

The secondary objectives of the study are to characterize the pharmacokinetics (PK) and PD of AVTX-006 in adults (aged 18-31 years) with active, moderate to severe complex lymphatic malformations; to characterize the pre- and post-treatment biomarker profile related to inhibition of mTORC1/2 and other related pathways; to evaluate the effect of AVTX-006 on the clinical and radiological manifestations of moderate to severe complex lymphatic malformations.

The primary endpoint of the study is incidence of adverse events (AEs), and changes in vital signs, ocular exam, cardiology tests, including echocardiogram (ECHO) and electrocardiogram (ECG), and clinical laboratory results.

The secondary endpoints of the study are: serum concentrations of AVTX-006 over time; changes from baseline in the levels of biomarkers related to mTOR and related pathways (e.g., phosphatidylinositol 3-kinase [PI3K], AKT); change from baseline to End of Treatment in radiologic assessment, if clinically indicated and/or clinical signs/symptoms of disease; change over time in Quality-of-life assessment, Karnofsky Performance Status and pain scale assessment.

### Example 1.2—Study Design

This is a Phase 1b, 2-cohort, open-label study in adults (aged 18-31 years) with active, moderate to severe complex lymphatic malformations. Approximately 10 subjects are planned to be enrolled in 2 cohorts. Following a screening period of up to 28 days, the first cohort of 5 subjects will receive AVTX-006 at a dose of 0.5 mg orally twice daily (approximately 12 hours apart) for 28 days. Based on evaluation of safety data, with an emphasis on any treatment-emergent Grade 3 or higher AEs in the first dosing cohort, and any available PK and PD data, the Safety Review Committee (SRC) will determine if the study proceeds to enrollment of a second cohort of 5 subjects. The second dose cohort will receive AVTX-006 at a dose of 1 mg orally twice daily (approximately 12 hours apart) for 28 days. After the initial procedures related to PK and PD assessments, clinic visits will be once a week during the treatment period. All subjects will be followed-up for 2 weeks after the last dose of AVTX-006.

There will be a washout period of at least 2 weeks prior to initiation of AVTX-006 for subjects receiving sirolimus (also known as rapamycin), mitogen-activated protein kinase (MEK) inhibitors, PIK3CA inhibitors, interferon alfa 2b, vascular endothelial growth factor receptor 3 (VEGFR-3) inhibitors, and/or other systemic agents targeting lymphatic malformations. There will be a washout period of at least 7 days prior to initiation of AVTX-006 for subjects receiving topical agents targeting lymphatic malformations. The investigator cannot discontinue the ongoing treatments as part of this study without obtaining the subject's informed consent and determining subject's eligibility.

Key study assessments include safety assessments, PK characterization of AVTX-006, evaluation of the PD profile (biomarkers related to mTOR and other pathways related to development of lymphatic malformations), and evidence of clinical activity using quality of life, pain scale assessment, Karnofsky Performance Status, and radiologic evaluations.

AVTX-006 will be studied for the first time in subjects with complex lymphatic malformations in this study. Therefore, the primary objective of this study is to assess the safety and tolerability of AVTX-006 at a dose of 0.5 mg twice daily in 5 subjects with complex lymphatic malformations, and at a dose of 1 mg twice daily in the next 5 subjects if the 0.5 mg dose is found to be tolerable in the first 5 subjects. This study will also assess the PK, PD, and efficacy profile of AVTX-006 as secondary objectives. This study will gather safety, PK, PD, and preliminary efficacy data to inform pivotal clinical trial design in the pediatric population. The study will take place at approximately 6 study sites in the United States within the Consortium of iNvestigators of Vascular AnomalieS (CaNVAS). Approximately 10 subjects aged 18 to 31 years with complex lymphatic malformations are planned to be enrolled. Although complex lymphatic malformations are more commonly observed in children (Cheng J, Liu B, Farjat A E, Routh J. National Characteristics of Lymphatic Malformations in Children: Inpatient Estimates and Trends in the United. States, 2000 to 2009. J Pediatr Hematol Oncol. 2018; 40(3):221-223), the current study is planned to be conducted in adults, as this is a first study in patients with complex lymphatic malformations. The data collected from adult subjects in this study will help in designing a pivotal clinical trial for the pediatric population. A screening log of study candidates will be maintained at each study site.

### Study Entry Criteria

Following assessment of study entry criteria, and completion of all screening assessments, the Patient Registration Form must be submitted to the Medical Monitor for review. Approval of the subject is required prior to 1st dose.

Inclusion Criteria Subjects must fulfill the following requirements to be eligible for the study:
1. Subject is 18 to 31 years of age (inclusive) at the time of consent. The date of signature of the informed consent is defined as the beginning of the screening period.
2. Subject has a verified diagnosis of any of the following complex lymphatic malformations: generalized lymphatic anomaly, Gorham syndrome, kaposiform lymphangiomatosis, microcystic lymphatic malformation, capillary lymphatico/venous malformation, abnormalities of the central conducting lymphatic channels, venous lymphatic malformation, and capillary lymphatic arterial venous malformation. Subjects with PTEN hamartoma tumor syndrome/vascular lesions may be considered with approval by the study medical monitor. Other moderate to severe vascular anomalies with associated lymphatic involvement (as described in the current ISSVA classification, ISSVA Classification of Vascular Anomalies. 2018.) will be considered, with approval by the study medical monitor.
3. Subject's complex lymphatic malformation is considered, in the opinion of the investigator, to be moderate to severe based on medical history, imaging, and/or disease-related complications requiring systemic control. Complications may include coagulopathy, chronic pain, recurrent cellulitis, ulceration, visceral and/or bone involvement, and cardiac dysfunction.
4. Subject has adequate liver function defined as:
   a. Total bilirubin (sum of conjugated and unconjugated)≤1.5×upper limit of normal (ULN)
   b. Aspartate transaminase/Alanine aminotransferase (AST/ALT)<5×ULN
   c. Serum albumin >2 g/dL
5. Subject has fasting low-density lipoprotein (LDL) of <160 mg/dL: subjects taking a cholesterol-lowering agent must be on a single medication and on a stable dose for at least 4 weeks prior to first dose of study drug.
6. Subject has adequate bone marrow function defined as:
   a. Peripheral absolute neutrophil count (ANC)>1000/μL
   b. Hemoglobin >8.0 g/dL (may receive red blood cell transfusions)
   c. Platelet count ≥50,000/μL (transfusion independent defined as not receiving a platelet transfusion within the 7-day period prior to enrollment through the screening period)
7. Subject has adequate renal function defined as: serum creatinine ≤1.5 mg/dL or calculated creatinine clearance of >50 mL/min according to the Cockcroft-Gault equation
8. Subject has agreed to and met the washout period as follows:
   a. At least 14 days prior to initiation of AVTX-006 if receiving sirolimus (also known as rapamycin), MEK inhibitors, PIK3CA inhibitors, interferon alfa 2b, VEGFR-3 inhibitors, and/or other systemic agents targeting lymphatic malformation
   b. At least 7 days prior to initiation of AVTX-006 if receiving topical agents targeting lymphatic malformation
      Note: Discontinuation of the ongoing treatments as part of this study should not be performed without obtaining the subject's informed consent and determining subject's eligibility.
9. Subject is non-pregnant and non-lactating (only for female).
10. Females of childbearing potential, and males with female partners of childbearing potential, who participate in the study agree to use a highly effective method of contraception throughout the study and for 28 days following the last dose of study drug. A highly effective method of birth control is defined as one that results in a low failure rate (ie, <1% per year) when used consistently and correctly, such as oral/injectable/inserted/implanted/transdermal contraceptives, condom with diaphragm, condom with spermicide, diaphragm with spermicide, intrauterine hormone-releasing system or intrauterine device (IUD), or sexual abstinence. Contraception is not required where at least 6 weeks has passed since sterilization, defined as females having undergone one of the following surgeries: hysterectomy, bilateral tubal ligation or occlusion, bilateral oophorectomy, or bilateral salpingectomy; and males who are vasectomized. Contraception is not required where females are postmenopausal (12 consecutive months of spontaneous amenorrhea and age ≥51 years).
11. Subject has a Karnofsky performance status of ≥50.
12. Subject has provided written informed consent for this study.
13. Subject is willing and able to comply with the protocol.

### Exclusion Criteria

The presence of any of the following criteria excludes a subject from the study:
1. Subject is, in the opinion of the investigator, mentally or legally incapacitated, or has significant emotional problems at the time of the screening visit that could interfere with the subject's participation or cooperation with the conduct of study evaluations.
2. Subject has a concurrent severe or uncontrolled medical disorder, which could compromise participation in the study (e.g. uncontrolled diabetes, severe infection, severe malnutrition, chronic liver or renal disease, active upper GI tract ulceration).
3. Subject has significant impairment of gastrointestinal function or gastrointestinal disease that may significantly alter the absorption of AVTX-006 (e.g. ulcerative disease, uncontrolled nausea, vomiting, diarrhea, malabsorption syndrome or small bowel resection). A gastric tube or nasogastric tube is permitted.
4. Subject has undergone a major surgery within 2 weeks prior to screening.
5. Subject has taken any medication that is a strong cytochrome P450 3A4 (CYP3A4) enzyme inducer or inhibitor within 2 weeks prior to first dose of study drug, or in the opinion of the Investigator, subject may require such medication during the study. See the Prohibited Therapies Section for the list of CYP3A4 inhibitors or inducers.
6. Subject is receiving chronic treatment with systemic steroids or another immunosuppressive agent, or in the opinion of the Investigator, subject may require such medication during the study. Subjects with endocrine deficiencies can receive physiologic or stress doses of steroids if necessary.
7. Subject has undergone myelosuppressive chemotherapy within 2 weeks, or radiation within 4 weeks prior to first dose of study drug.
8. Subject has received hematopoietic growth factors, antineoplastic agents, or enzyme-inducing anticonvulsants within 2 weeks prior to first dose of study drug, or in the opinion of the Investigator, subject may require such medication during the study.
9. Subject has a known history of uncontrolled hypertension, cardiovascular disease, advanced arteriosclerosis, structural cardiac abnormality, cardiomyopathy, serious heart rhythm abnormalities, coronary artery disease, cardiac conduction problems, heart failure, exercise related cardiac events including syncope and pre-syncope, or a known family history of sudden cardiac death or ventricular arrhythmia.
10. Subject has any clinically significant abnormality on 12-lead ECG and/or ECHO at screening, such as serious arrhythmia, cardiac conduction problems, or other abnormalities deemed to be a potential safety issue.
11. Subject has known human immunodeficiency virus (HIV).
12. Subject has known or suspected intolerance or hypersensitivity to the investigational product(s), closely related compounds, or any ingredients of the investigational product.
13. Subject has a history of significant, multiple and/or severe allergies, or has had an Anaphylactic reaction or significant intolerability to prescription or nonprescription drugs or food.
14. Subject has used a systemic investigational product within 2 weeks prior to first dose of study drug or a topical investigational product within 7 days prior to first dose of study drug.
15. Subject has a history of difficult blood draws that in the opinion of the investigator would potentially compromise the study conduct.
16. Subject has any clinically significant laboratory abnormality at screening, as described by the inclusion criteria above, or as determined by the Investigator.
17. Subject has received treatment with erlotinib, or other epidermal growth factor receptor (EGFR) inhibitors within 4 weeks prior to the first dose of study drug, or in the opinion of the Investigator, subject may require such medication during the study.
18. Subject has received treatment with a medication that has the potential to prolong the QT interval within 1 week prior to first dose of study drug, or in the opinion of the Investigator, subject may require such medication during the study.

### Screen Failures

Subjects who fail inclusion and/or exclusion criteria may be rescreened for the study with the prior approval of the Medical Monitor. In the event of a rescreening, the first screening visit will be entered into the electronic case report form (eCRF) as the screening visit and the repeat assessments entered into the eCRF as an unscheduled visit. First dose of study drug should occur within 28 days of re-screening.

### Premature Subject Withdrawal

All subjects will be advised that they are free to withdraw from participation in this study at any time, for any reason, and without prejudice. Every reasonable attempt should be made by the investigator to keep subjects in the study; however, subjects must be withdrawn from the study if they withdraw consent to participate. Investigators must attempt to contact subjects who fail to attend scheduled visits by telephone or other means to exclude the possibility of an AE being the cause of withdrawal. Should this be the cause, the AE must be documented, reported, and followed.

Subjects can decline to continue receiving study drug at any time during the study. If this occurs, the investigator is to discuss with the subject the completion of the Follow-up/Early Termination Visit within 3 days following the last dose of study drug Subjects should also be encouraged to return for the Safety Follow-up visit 7-10 days post last dose and to participate in the Late Follow-up phone call 2 weeks (±2 days) post last dose. If the subject refuses these visits/procedures associated with these visits, data on concomitant medications and AEs will be collected if the subject agrees. Data on concomitant medications and AEs can be collected via a telephone call if the subject refuses an in-person visit.

Withdrawal of consent for a study means the subject does not wish to receive further protocol-required treatment or procedures, and the subject does not wish to or is unable to continue further study participation. Subject data up to withdrawal of consent will be included in the analysis of the study, and where permitted, publicly available data can be included after withdrawal of consent. A follow-up call will be made 14 days (±2 days) after the subject's last dose of AVTX-006. Subjects can decline to participate in the telephone call.

The Sponsor reserves the right to request the withdrawal of a subject due to protocol deviations or other reasons.

The investigator also has the right to withdraw subjects from the study at any time for any reason. If a subject is withdrawn before completing the study, the subject should be followed-up as instructed in the Schedule of Assessments (Table 1). The reason for withdrawal must be determined by the investigator and recorded in the subject's medical record and in the electronic case report form (eCRF). If a subject is withdrawn for more than 1 reason, each reason should be documented in the source document and the most clinically relevant reason should be entered in the eCRF.

Reasons for discontinuation include but are not limited to:
Lack of effect, including worsening of symptoms, disease progression, or lack of improvement
Adverse event
Death
Non-compliance with study procedure/protocol
Decision by investigator
Decision by Sponsor
Withdrawal of consent by subject
Lost to follow-up
Other (specify). For example, pregnancy.

### Subject Replacement Criteria

Subjects who withdraw from the study for reasons other than drug toxicity may be replaced with the prior approval of the Medical Monitor.

TABLE 1
Schedule of Assessments

| | | | | | | | End of | | |
|---|---|---|---|---|---|---|---|---|---|
| | | Treatment Period | | | | | Treatment/ | | |
| | Screening | Day 1 | Day | Day | Day | Day | Early | Safety | Late |
| | Visit^{1, 2} | (Baseline)³ | 2 | 8 | 15 | 22 | Termination⁴ | Follow-up⁵ | Follow-up⁶ |
| Visit Window (Days) | | | | ±2 | ±2 | ±2 | +3 | | |
| Informed Consent² | X | | | | | | | | |
| Inclusion/Exclusion | X | X | | | | | | | |
| Review | | | | | | | | | |
| Demographics | X | | | | | | | | |
| Medical and | X | | | | | | | | |
| Medication History, | | | | | | | | | |
| including baseline | | | | | | | | | |
| disease assessment | | | | | | | | | |
| Physical Exam | X | | | | | | X | X | |
| Karnofsky | X | X | | | | | X | | |
| Performance Status | | | | | | | | | |
| Height | X | | | | | | | | |
| Vital Signs, including | X | X | X | X | X | X | X | X | |
| weight⁷ | | | | | | | | | |
| 12-lead ECG⁸ | X | X | | X | | | X | | |
| ECHO | X | X | | | X | | X | | |
| Cardiac Labs: NT- | X | X | | | X | | X | | |
| proBNP, troponin, | | | | | | | | | |
| creatine kinase | | | | | | | | | |
| Radiologic disease | X | | | | | | X | | |
| assessment⁹ | | | | | | | | | |
| Slit lamp examination¹⁰ | X | | | | X | | X | X | |
| Hematology¹¹ | X | X | | X | X | X | X | X | |
| Serum chemistry¹² | X | X | | X | X | X | X | X | |
| Coagulation panel¹³ | X | X | | X | X | X | X | X | |
| Lipid panel¹⁴ | X | | | | X | | X | X | |
| Urinalysis¹⁵ | X | X | | X | X | X | X | X | |
| Serum pregnancy test¹⁶ | X | | | | | | | | |
| Urine pregnancy test¹⁶ | | X | | | | | X | | |
| Pharmacokinetic | | X | X | X | X | X | X | | |
| sampling¹⁷ | | | | | | | | | |
| Biomarkers¹⁷ | | X | X | X | X | X | X | | |
| Genotyping¹⁸ | X | | | | | | | | |
| QOL and Pain Scale | | X | | | X | | X | | |
| Assessment¹⁹ | | | | | | | | | |
| Adverse event | X | X | X | X | X | X | X | X | X |
| monitoring²⁰ | | | | | | | | | |
| Concomitant | X | X | X | X | X | X | X | X | X |
| medications | | | | | | | | | |
| Study drug | | X | X | X | X | X | | | |
| administration²¹ | | | | | | | | | |
| Subject diary²² | | X | X | X | X | X | | | |
| Investigator | | | | | | | X | | |
| Assessment of | | | | | | | | | |
| Disease²³ | | | | | | | | | |
| Taste Survey²⁴ | | | | | | | X | | |
| Patient Registration | X | | | | | | | | |
| Form²⁵ | | | | | | | | | |

¹Screening visit to occur within 28 days prior to first dose of study drug.
²If more than 28 days have elapsed between the screening visit and the planned Day 1 of treatment, the subject must be re-consented. Additionally, all screening procedures must be repeated with the exception of demography, genotyping and radiologic disease assessment. Applicable inclusion and exclusion criteria must be reassessed using the results received. Dosing on Day 1 must occur within 28 days of any re-screening assessments.
³If the screening visit occurs within 3 days of the Baseline Day 1 visit, the following procedures need not be repeated: pre-dose ECG, ECHO, cardiac labs, and urinalysis.
Note:
Screening and Baseline visits can also occur on the same day, in which instance procedures collected on both days will only be collected once. For pregnancy testing, only the serum test will be collected; the urine test does not need to be performed.
⁴End of Treatment/Early Termination visit to occur within 3 days following the last dose of study drug. In case of early termination, if the subject refuses the scheduled visit/procedures, every effort will be made to collect data on concomitant medications and adverse events (if the subject agrees). This data may be collected via telephone call if subject refuses an in person visit.
⁵A safety follow-up visit will occur 7-10 days after the last dose of study drug.
⁶A late follow-up telephone call will be made 2 weeks (±2 days) after the subject's last dose of study drug.
⁷Vital signs include pulse, blood pressure, respiratory rate, temperature, and weight.
⁸For Baseline visit, ECG to be performed prior to dose and 4 hours (±2 hours) following the first dose. On Day 8, ECG is to be performed within 4 hours (±2 hours) following dosing. ECG at End of Treatment/Early Termination to be done at any time during the visit.
⁹Radiologic disease assessment will be done if clinically indicated, at the discretion of the investigator. The type of imaging to be performed is at the discretion of the investigator, based on the disease type and the subject's clinical manifestations.
¹⁰Slit lamp examination will include assessment of cornea, anterior chamber, lens, and anterior vitreous. For screening/Day 1 visits, a single examination may be done prior to 1st dose.
¹¹Hematology includes white blood cell count, differential count, hemoglobin, hematocrit, and platelet count.
¹²Serum chemistry includes sodium, potassium, chloride, bicarbonate, glucose, blood urea nitrogen, creatinine, alanine aminotransferase, aspartate aminotransferase, alkaline phosphatase, total bilirubin, calcium, magnesium, phosphorous, uric acid, total protein, and albumin. Creatinine clearance will be calculated (according to Cockcroft-Gault equation).
¹³Coagulation panel includes partial thromboplastin time, international normalized ratio, fibrinogen, and d-dimer.
¹⁴Fasting lipid panel includes total cholesterol, HDL, LDL, and triglycerides.
¹⁵Urinalysis includes pH, specific gravity, dipstick determinations of protein, blood, glucose and ketones.
¹⁶For females of childbearing potential.
¹⁷Blood sampled for PK and PD/biomarkers will be collected prior to the first dose, and 1 hour (±15 min), 2 hours (±15 min), 4-6 hours, and optional 8-12 hours post dose on Day 1, prior to morning dose on Day 2, and anytime on Days 8, 15, 22 and End of Treatment/Early Termination (Table 3).
¹⁸Prior genotyping information will be documented for subjects with known mutational status. Optional genotyping will be offered to subjects with archival tissue that has not been previously assessed, and to subjects willing to provide new tissue samples for genotyping analysis. Archival tissue or new tissue samples for genotyping analysis will be accepted at any time during the subject's time in the study. In addition, at the screening visit, subjects will be given the opportunity to provide an optional blood sample for potential future development of a methodology for genotyping utilizing DNA circulating in peripheral blood and to further scientific knowledge in genetic mutations associated with lymphatic malformations.
¹⁹SF-36 and pain scale assessment using a Visual Analogue Scale (VAS) will be performed.
²⁰Adverse events will be collected from the time the informed consent is signed through the Safety Follow-up visit, and serious adverse events will be collected from the time of informed consent through the Late Follow-up phone call.
²¹AVTX-006 will be administered starting from Day 1 to Day 28 twice daily. On Study Day 1, study drug is to be administered after all assessments are completed, except where specified otherwise. On Day 2, study drug is to be administered at the study site, following PK sample collection.
²²At Baseline, all subjects will be provided a subject diary to note timing and volume of each dose. Subjects will be asked to bring this diary during each visit.
²³Investigator will assess disease status compared to Baseline and rate the subject's current disease status as no change from baseline, radiological improvement from baseline, radiological worsening from baseline, clinical signs/symptoms improved from baseline and/or clinical signs/symptoms worsened from baseline.
²⁴Subjects will be asked to complete a taste survey describing the taste of AVTX-006, how long the taste lasted, and whether the taste would have a negative impact on their willingness to take AVTX-006 long term.
²⁵Following completion of screening procedures, the Patient Registration Form must be submitted to the Medical Monitor for review. Approval of the subject is required prior to 1st dose.
Abbreviations: ECG = electrocardiogram; ECHO = echocardiogram; HDL = high density lipoprotein; LDL = low density lipoprotein; QOL = quality of life; NT-proBNP: N-terminal-pro hormone BNP.

### Example 1.3—Treatments

### Identification of Investigational Product, Dose and Mode of Administration

AVTX-006, trans-4-[4-Amino-5-(7-methoxyl-1H-indol-2-yl)-imidazo[5,1-f][1,2,4]triazine-7-yl]-cyclohexanecarboxylic acid, tromethamine salt, is the investigational product that will be used in this study. It is in the form of an oral solution. The dose is 0.5 mg for the first cohort of 5 subjects, and 1 mg for the second cohort of 5 subjects. The frequency of dosing is twice daily (approximately 12 hours apart) for 28 days. The mode of administration is oral.

Treatments Administered

The first cohort of 5 subjects will receive AVTX-006 at a dose of 0.5 mg orally twice daily (approximately 12 hours apart) for 28 days. Following a review by the SRC, if there are no clinically important safety findings in the first dosing cohort, the second cohort of 5 subjects will receive AVTX-006 at a dose of 1 mg orally twice daily (approximately 12 hours apart) for 28 days.

Subjects will be provided with dosing cups and syringes (needleless) to ensure accurate dosing. A new dosing cup and a new syringe should be used each time the subject takes their dose of study drug. Study drug is to be taken twice a day, with doses approximately 12 hours apart. If a subject forgets to take a dose at the designated time, it may be taken late provided it is within 4 hours of the original planned dose time. Doses should not be taken any closer than 8 hours apart. If greater than 4 hours from the original dose time, the dose should be skipped and recorded as a missed dose.

### Dispensing and Storage

Bulk investigational product in powder form must be stored at controlled room temperature 15-25° C. (59-77° F.) in the original container, protected from light and in a secure area that is temperature monitored. Prior to dispensing to the subject, the powder is to be reconstituted with sterile water for injection at a concentration of 0.1 mg/mL. Once reconstituted, drug product is to be stored at 2-8° C., with light protection. An oral syringe will be used to ensure accurate dosing.

### Blinding and Unblinding Treatment Assignment

This is an open-label study.

Selection of Doses in the Study

The doses proposed for this study were selected after modeling and simulation of the human PK of CERC-006 using data reported by Mateo et al (Mateo J, Olmos D, Dumez H, et al. A first in man, dose-finding study of the mTORC1/mTORC2 inhibitor OSI-027 in patients with advanced solid malignancies. Br J Cancer. 2016114(8):889-896) and comparing the PK/PD response for sirolimus in patients with complicated vascular anomalies published by Adams et al (Adams et al, Pediatrics (2016)) and Mizuno et al (Mizuno T, Emoto C, Fukuda T, et al. Model-based precision dosing of sirolimus in pediatric patients with vascular anomalies. Eur J Pharm Sci. 2017; 109S:S124-S131). The goal was to estimate doses that would produce effective trough plasma concentrations of CERC-006 in relation to pharmacological target potency (mTOR complex inhibition) using the PK/PD response achieved by sirolimus in this patient population as a benchmark. Proposed doses of CERC-006 that would produce and maintain free plasma concentrations exceeding the IC₅₀ for mTOR complex inhibition by 2-fold were estimated by linear extrapolation of Cₘₐₓ and minimum concentration (Cₘᵢₙ) from the modeling and simulation PK profile of CERC-006. A total daily dose of 1.0 mg, divided every 12 hours, would produce total and free trough concentrations of approximately 27 ng/mL (66 nM) and 4 ng/mL (10 nM), approximately 2-fold above the in vitro IC₅₀ for mTOR complex inhibition (4 nM) at the 50^{th} percentile. The additional dose proposed, 2.0 mg/day, is necessary to ensure potential therapeutic benefit while preserving safety and providing a range of plasma concentrations to guide future PK/PD modeling efforts.

### Dose Adjustment Criteria

No dose adjustments are planned for this study.

### Prior Therapies

Prior therapies includes all treatments received within 28 days of the date of first dose of investigational product. Prior treatment information must be recorded on the appropriate eCRF page.

### Concomitant Therapies

Concomitant therapies refer to all therapies taken on or after first dose of study drug through the last visit. Concomitant therapy information must be recorded on the appropriate eCRF page.

### Permitted Therapies

Patients are treated for all intercurrent medical conditions at the discretion of the investigator in accordance with community standards of medical care. Subjects with endocrine deficiencies can receive physiologic or stress doses of steroids if necessary.

Prohibited Therapies

After completion of the screening visit and determination that the subject qualifies for the study, ongoing treatment for lymphatic malformation will be discontinued (if applicable). There will be a washout period of at least 2 weeks prior to initiation of AVTX-006 for subjects receiving sirolimus (also known as rapamycin), MEK inhibitors, PIK3CA inhibitors, interferon alfa 2b, VEGFR-3 inhibitors, and/or other systemic agents targeting lymphatic malformations. There will be a washout of at least 7 days prior to initiation of AVTX-006 for subjects receiving topical agents targeting lymphatic malformations. The investigator cannot discontinue the ongoing treatments as part of this study without obtaining the subject's informed consent and determining subject's eligibility.

Any new therapy (topical or systemic), other than AVTX-006, designed to treat the subject's lymphatic malformation is prohibited during the 28-day treatment period. Note: use of topical steroids is not restricted during the study.

During the study, new initiation of investigational compounds is prohibited. Vaccines that are considered by the investigator during the clinical trial should be discussed with the medical monitor prior to administration.

Treatment with hematopoietic growth factors and antineoplastic agents is prohibited within 2 weeks prior to the first dose of study drug and during the 28-day treatment period. Treatment with systemic steroids or other immunosuppressive agents is prohibited during the 28-day treatment period. Subjects with endocrine deficiencies can receive physiologic or stress doses of steroids if necessary.

A list of prohibited CYP3A4 inhibitors, CYP3A4 inducers, and enzyme inducing anticonvulsants includes but is not limited to:
CYP3A4 inhibitors: Subjects should not be currently receiving strong inhibitors of CYP3A4 and should not have received these medications within 2 weeks prior to the first dose of AVTX-006 through last dose of study drug. These medications include but are not limited to:
   Macrolide antibiotics: clarithromycin, telithromycin, erythromycin,
   troleandomycin.
   Gastrointestinal prokinetic agents: cisapride, metoclopramide.
   Antifungals: itraconazole, ketoconazole, fluconazole (doses >200 mg/day),
   voriconazole, clotrimazole.
   Calcium channel blockers: verapamil, diltiazem, nicardipine.
   Other drugs: rifampin, bromocriptine, cimetidine, danazol, cyclosporine oral
   solution, lansoprazole.
   Grapefruit juice.
CYP3A4 inducers: Subjects should also avoid strong inducers of CYP3A4 and should not have received these medications within 2 weeks prior to the first dose of AVTX-006 through last dose of study drug. These medications include but are not limited to:
   Anticonvulsants: carbamazepine, phenobarbital, phenytoin.
   Antibiotics: rifabutin, rifapentine.
   Herbal preparations: St. John's Wort (Hypericum perforatum, hypericine).
Enzyme inducing anticonvulsants: Subjects should not be taking enzyme-inducing anticonvulsants and should not have received these medications within 2 weeks prior to the first dose of AVTX-006 through last dose of study drug, as these subjects may experience different drug disposition. These medications include but are not limited to: Carbamazepine, Felbamate, Phenobarbitol, Phenytoin, Primidone, and Oxcarbazepine.

Subjects should not be receiving treatment with erlotinib, or other epidermal growth factor receptor inhibitors within 4 weeks prior to first dose of AVTX-006 through last dose of study drug. Subjects should not be receiving treatment with a medication that has the potential to prolong the QT interval within 1 week prior to first dose of AVTX-006 through last dose of study drug.

At the discretion of the Sponsor, subjects receiving excluded therapies during the study may be ineligible for continuation in the study.

Treatment after End of Study

Following discontinuation from the study, subjects may be treated at the discretion of the investigator.

### Example 1.4—Study Procedures

For the timing of assessments and procedures throughout the study, refer to the Schedule of Assessments (Table 1). Throughout the study, every reasonable effort should be made by study personnel to follow the timing of assessments and procedures in the schedule of events for each subject. If a subject misses a study visit for any reason, the visit should be rescheduled as soon as possible. Visits should not be skipped during the study.

### Study Duration

The sequence and maximum duration of the study periods will be as follows. The screening period will be up to 28 days. The treatment period will be 28 days. The follow-up period will be 14 days (±2 days) after the last dose of the investigational product. The maximum study duration for each subject will be approximately 10 weeks.

### Medical History

A medical and surgical history will be taken at Screening, including a baseline assessment of clinical signs/symptoms associated with the subject's complex lymphatic malformation. All significant medical history findings that have been present or active within the 5 years prior to screening will be entered into the eCRF. Medical history findings that have not been present within the 5 years prior to screening will be recorded if deemed clinically relevant by the Investigator to the conduct of the study.

### Safety Assessments

Safety and tolerability assessments will include the frequency and severity of AEs and evaluation of vital signs, ocular exam (slit lamp examination), clinical laboratory results including renal function, ECG, ECHO, and imaging results.

### Clinical Laboratory Tests to be Performed

Samples for the following clinical laboratory tests will be collected at the time points specified in the Schedule of Assessments (Table 1).
Hematology tests include Hemoglobin, hematocrit, platelet count, and white blood cell count including differential.
Serum chemistry tests include sodium, potassium, glucose, bicarbonate, glucose, blood urea nitrogen, creatinine, alanine aminotransferase, aspartate aminotransferase, alkaline phosphatase, total bilirubin, calcium, magnesium, phosphorous, uric acid, total protein, and albumin. Creatinine clearance will be calculated (according to Cockcroft-Gault equation).
Coagulation panel tests include partial thromboplastin time, international normalized ratio, fibrinogen, and d-dimer.
A serum/urine pregnancy test will be administered for females of childbearing potential.
Lipid panel (fasting) tests include total cholesterol, high density lipoprotein, LDL, and triglycerides.
Cardiac tests include N-terminal pro-brain natriuretic peptide (NT-proBNP), troponin, and
creatine kinase.
Urinalysis tests include pH, specific gravity, dipstick determinations of protein, blood, glucose and ketones.
Laboratory specimens will be collected and analyzed locally at each clinical site.

### Sampled Blood Volume

The sampled blood volume is shown in Table 2.

TABLE 2
Sampled Blood Volume per Subject

| | Volume per | Sample | |
|---|---|---|---|
| Type | sample | number | Total |
| Hematology | 3 mL | 7 | 21 mL |
| Coagulation panel | 3 mL | 7 | 21 mL |
| Serum chemistry | 3.5 mL | 7 | 24.5 mL |
| Serum pregnancy | 2 mL | 1 | 2 mL |
| Lipid panel | 3.5 mL | 4 | 14 mL |
| Pharmacokinetics | 2 mL | 10 | 20 mL |
| Pharmacodynamics/Biomarkers | 4 mL | 10 | 40 mL |
| Genotyping* | 5 mL | 1 | 5 mL |
| NT-proBNP, creatinine kinase | 5 mL | 4 | 20 mL |
| and troponin | | | |
| Total (up to) | | | 168.0 mL |

*Blood sample for genotyping is optional.
These parameters exclude rescreening laboratory samples, additional tests to monitor potential adverse events, confirmatory pregnancy tests, or for any unscheduled visits. The volumes provided are an estimate and may vary depending on local lab requirements.

### Evaluation of Laboratory Values

The normal ranges of values for the laboratory assessments in this study will be provided by the applicable laboratory facility. They will be regarded as the reference ranges on which decisions will be made for the specific site.

If a laboratory value is out of the reference range, it is not necessarily clinically relevant. The investigator must evaluate the out-of-range values and record his/her assessment of the clinical relevance in the subject's source documentation. Abnormal laboratory values may be considered AEs if they are clinically significant, if they require an intervention, or if they change the administration of study drug.

All laboratory values which, in the investigator's opinion, show clinically relevant or pathological changes during or after termination of the treatment are to be discussed with the Medical Monitor, as necessary, and reported as AEs and followed.

### Clinical Examination: Vital Signs

Blood pressure, pulse rate, respiratory rate, temperature, and body weight will be measured at times specified in the Schedule of Assessments (Table 1). Additional blood pressure and pulse rate measurements may be performed, as determined by the investigator, to ensure appropriate monitoring of subject safety and accurate recording of vital sign measurements. Any changes from baseline which are deemed clinically significant by the investigator are to be recorded as an AE and followed.

### Clinical Examination: Electrocardiogram

A standard 12-lead ECG will be performed after the subject has been supine for approximately 5 minutes, at times specified in the Schedule of Assessments (Table 1). All ECG recordings will be identified with the subject number, date, and time of the recording and a copy will be included with the subject's source documentation. All ECG values which, in the investigator's opinion, show clinically relevant or pathological changes during or after termination of the treatment are to be discussed with the Medical Monitor and reported as AEs and followed.

### Clinical Examination: Physical Examination

A physical examination will be performed at times specified in the Schedule of Assessments (Table 1). Height will be measured at Screening. Any clinically significant physical examination findings post-screening are to be reported as AEs and followed.

### Clinical Examination: Electrocardiography

An ECHO assessment will be done at time points specified in the Schedule of Assessments (Table 1). Any clinically significant ECHO findings post screening are to be discussed with the Medical Monitor and reported as AEs and followed.

### Clinical Examination: Slit Lamp Examination

Slit lamp examination will be done at time points specified in the Schedule of Assessments (Table 1). Any clinically significant ophthalmologic findings post screening are to be reported as AEs and followed. Slit lamp examination will include the following parameters:
Cornea: Clear? Epithelial disruptions? Stromal opacities? Endothelial lesions?
Anterior chamber: Deep? Cell or flare?
Lens: Clear? Nuclear, cortical or subcapsular cataract?
Anterior vitreous: Inflammation? Hemorrhage? Pigmented cells?

### Clinical Examination: Adverse Events

The investigator is responsible for the detection and documentation of events meeting the criteria and definition of an AE or SAE described previously. At each visit, the subject will be allowed time to spontaneously report any issues since the last visit or evaluation.

Any clinically relevant observations made during the visit will also be considered AEs.

### Pharmacokinetics

Blood draws for PK analysis will be performed in accordance with Table 1 and Table 3. Details of PK sample collection and processing can be found in the site and/or study laboratory manual. Plasma samples will be analyzed via validated methods for AVTX-006 concentrations and its metabolites.

TABLE 3
Pharmacokinetic and Pharmacodynamic/Biomarker Sample Collection Schedule

| | | Day 1 | | | | | | | | |
|---|---|---|---|---|---|---|---|---|---|---|
| | | 1 | 2 | 4 | 8 hours | 24 hours | Day | Day | Day | EOT/ |
| | Baseline^{a} | hour | hours | hours | (Optional) | (Day 2)^{b} | 8 | 15 | 22 | ET |
| Window | | ±15 | ±15 | +2 | +4 | | | | | |
| | | min | min | hours | hours | | | | | |
| PK | X | X | X | X | X | X | X | X | X | X |
| PD/ | X | X | X | X | X | X | X | X | X | X |
| Biomarker | | | | | | | | | | |

^{a}Baseline: Day 1, prior to first dosing.
^{b}As close as possible to 24 hours following the 1st dose of study drug on Day 1.
Note:
Blood sampled for PK and PD/biomarkers will be collected prior to the first dose, and 1 hour (±15 min), 2 hours (±15 min), 4-6 hours, and optional 8-12 hours post dose on Day 1, prior to morning dose on Day 2, and anytime on Days 8, 15, 22 and EOT/ET.
EOT: end of treatment;
ET: Early termination;
PD: pharmacodynamics;
PK: pharmacokinetics.

### Pharmacodynamics

The PD will be determined by measuring levels of biomarkers (such as p4EBP1, pS6 [Ribosomal protein S6 kinase beta-1], pPRAS40 [proline-rich Akt substrate of 40 kDa], pAKT, Cleaved Caspase 3 and Ki67) related to mTOR and other pathways associated with lymphatic disease process and regulation (Table 1 and Table 3).

### Genotyping

Prior genotyping information will be collected for subjects with known mutational status. Subjects who have not had prior genotyping performed will be offered one of the following as optional testing (to be performed locally, at any time during the study):
Genotyping analysis on previously archived tissue
Genotyping analysis on a new tissue sample if no archival tissue exists

In addition, subjects will be asked to provide an optional blood sample at the screening visit for potential future development of a methodology for genotyping utilizing DNA circulating in peripheral blood and to further scientific knowledge in genetic mutations associated with lymphatic malformations.

### Efficacy

The efficacy of AVTX-006 will be evaluated by radiologic and clinical assessment at the end of the treatment period and quality-of-life assessment using 36-Item Short Form Health Survey (SF-36), visual analog scale (VAS), and Karnofsky Performance Status during the treatment period (Table 1).

### Radiologic Disease Assessment

Radiologic disease assessment will be done at the time points specified in the Schedule of Assessments (Table 1). Of note, radiologic disease assessment will only be done if clinically indicated, at the discretion of the investigator. The type of imaging to be performed is at the discretion of the investigator, based on the disease type and the subject's clinical manifestations.

### Investigator Assessment of Disease

At the End of Treatment/Early Termination visit, the investigator will be asked to assess the subject's disease status compared to Baseline as:
No change from baseline
Radiological improvement from baseline
Radiological worsening from baseline
Clinical signs/symptoms improved from baseline
Clinical sign/symptoms worsened from baseline

If a change from baseline was observed, a descriptive characterization of that change will be provided.

### SF-36 Questionnaire

Quality of life of subjects will be evaluated using the SF-36 questionnaire at the time points specified in the Schedule of Assessments (Table 1). The SF-36 measures eight scales: physical functioning, role physical, bodily pain, general health, vitality, social functioning, role emotional, and mental health (tins L, Carvalho F M. SF-36 total score as a single measure of health-related quality of life: Scoping review. SAGE Open Med. 2016:4:2050312116671725.).

### Visual Analog Scale

Pain scale assessment will be done using a 100 mm VAS from no pain (0 mm) to worst pain (100 mm) at the time points specified in the Schedule of Assessments (Table 1).

Karnofsky Performance Status

Functional status of subjects will be evaluated using Karnofsky Performance Status (Table 4) at the time points specified in the Schedule of Assessments (Table 1). Subjects must have a Karnofsky Performance Status of ≥50 to enter the study.

TABLE 4
Karnofsky Performance Status

| Condition | Percentage | Comments |
|---|---|---|
| A: Able to carry on | 100 | Normal, no complaints, no |
| normal activity and to | | evidence of disease. |
| work. No special care | 90 | Able to carry on normal activity, |
| is needed. | | minor signs or symptoms |
| | | of disease. |
| | 80 | Normal activity with effort, some |
| | | signs or symptoms of disease. |
| B: Unable to work. Able | 70 | Cares for self, unable to carry |
| to live at home, care for | | on normal activity or to do |
| most personal needs. A | | active work. |
| varying degree of | 60 | Requires occasional assistance |
| assistance is needed. | | but is able to care for most |
| | | of his needs. |
| | 50 | Requires considerable assistance |
| | | and frequent medical care. |
| C: Unable to care for | 40 | Disabled, requires special care and |
| self. Requires equivalent | | assistance. [In bed more than 50% |
| of institutional or | | of the time]. |
| hospital care. Disease | 30 | Severely disabled, hospitalization |
| may be progressing | | is indicated although death not |
| rapidly. | | imminent. [Almost completely |
| | | bedfast]. |
| | 20 | Hospitalization necessary, very |
| | | sick, active supportive treatment |
| | | necessary. [Totally bedfast |
| | | and requiring extensive nursing |
| | | care by professionals and/or |
| | | family]. |
| | 10 | Moribund, fatal processes |
| | | progressing rapidly. |
| | | [Comatose or barely |
| | | arousable]. |
| | 0 | Dead |

Péus D, Newcomb N, Hofer S. Appraisal of the Karnofsky Performance Status and proposal of a simple algorithmic system for its evaluation. BMC Med Inform Decis Mak. 2013; 13: 72.

### Example 1.5—Adverse Events

### Adverse Event Collection

An AE is defined as any untoward medical occurrence in a patient or clinical investigation subject administered a pharmaceutical product that does not necessarily have a causal relationship with the product. An AE can therefore be any unfavorable and unintended sign (including a new, clinically important abnormal laboratory finding), symptom, or disease, temporally associated with the product, whether or not related to the product. An AE will be considered treatment-emergent if it occurs after the first dose of investigational product through the Safety Follow-up visit (i.e., 7-10 days post last dose of investigational product).

All AEs are collected from the time the informed consent is signed through the Safety Follow-up visit (7-10 days post last dose). SAEs are collected from the time of informed consent is signed through the Late Follow-up phone call (2 weeks±2 days post last dose) (Table 1). This includes events occurring during the screening phase of the study, regardless of whether investigational product is administered. Note: Clinically significant observations noted during screening procedures (labs, physical examination, vital signs, etc.) should be entered as medical history. Only if the clinically significant observation is clearly related to the performance of a screening procedure should it be entered as an AE (e.g., a hematoma because of drawing blood for screening labs). Where possible, a diagnosis rather than a list of symptoms should be recorded. If a diagnosis has not been made, then each symptom should be listed individually. All AEs should be captured on the appropriate AE pages in the eCRF and in source documents.

All AEs must be followed to closure, regardless of whether the subject is still participating in the study. Closure indicates that an outcome is reached, stabilization is achieved (ie, the investigator does not expect any further improvement or worsening of the event), or the event is otherwise explained. When appropriate, medical tests and examinations are performed so that resolution of an event(s) can be documented.

Lack of effect, including worsening of symptoms, disease progression, or lack of improvement, should not be recorded as an AE unless it meets the definition of the criteria for an SAE. AE that changes in severity over time should be recorded in the eCRF once at the highest severity with two exceptions:
Worsening of non-lymphatic malformation related pre-treatment events after initiation of investigational product must be recorded as new AEs. Eg, if the subject experiences mild, intermittent headaches prior to dosing with investigational product; however, the headache intensity increases to moderate after the first dose of investigational product, a new AE of moderate intermittent headaches is to be recorded in the source documents and eCRF.
An AE which begins as a non-serious event, which later meets the definition of an SAE, should be entered once for the non-serious portion of the AE, and then be re-recorded as a new event with the start date the day it became serious.

### Severity of Adverse Events

The medical assessment of clinical severity of an AE will be determined using the definitions outlined in Common Terminology Criteria for Adverse Events (CTCAE), Version 5.0 (Published Nov. 27, 2017 by the US Department of Health and Human Services, National Institutes of Health, National Cancer Institute). Grade 1 is defined as Mild; asymptomatic or mild symptoms; or clinical or diagnostic observations only; or intervention not indicated. Grade 2 is defined as Moderate; or minimal, local or non-invasive intervention indicated; or limiting age-appropriate instrumental activities of daily living (ADL). Grade 3 is defined as Severe or medically significant but not immediately life-threatening; or hospitalization or prolongation of hospitalization indicated; or disabling; or limiting self-care ADL. Grade 4 is defined as Life-threatening consequences; or urgent intervention indicated. Grade 5 is death related to AE.

The above grading guidelines should be used whenever possible. For AEs that cannot be graded by the use of CTCAE, the severity should be graded using mild (Grade 1), moderate (Grade 2), severe (Grade 3), life threatening (Grade 4), and fatal (Grade 5).

Please refer to the above-referenced CTCAE document for full description of CTCAE terms and instrumental and self-care ADLs. It is important to distinguish between severe AEs and SAEs. Severity is a classification of intensity whereas an SAE is an AE that meets serious criteria.

### Relationship Categorization

A physician investigator must make the assessment of relationship to investigational product for each AE. The investigator should decide whether, in his or her medical judgment, there is a reasonable possibility that the event may have been caused by the investigational product. If there is no valid reason for suggesting a relationship, then the AE should be classified as “not related”. Otherwise, the AE should be categorized per the guidelines below. The causality assessment must be documented in the source document and the eCRF (Table 5).

TABLE 5
Assessment of Relationship to Investigational Product

| Relationship | Description |
|---|---|
| Not | Exposure to investigational product has not occurred. |
| Related | OR |
| | The administration of investigational product and |
| | the occurrence of the AE are not reasonably |
| | related in time. |
| | OR |
| | The AE is considered likely to be related to an |
| | etiology other than the use of the investigational |
| | product, that is, there are no facts/evidence or |
| | arguments to suggest a causal relationship to the |
| | investigational product. |
| Possibly | The administration of the investigational product |
| Related | and the occurrence of the AE are reasonably |
| | related in time. |
| | AND |
| | The AE could not be explained equally well by |
| | factors or causes other than exposure to |
| | investigational product. |
| Probably | The administration of the investigational product |
| Related | and the occurrence of the AE are reasonably |
| | related in time. |
| | AND |
| | The AE is more likely explained by exposure |
| | to investigational product than by other |
| | factors or causes. |

AE = adverse event.

### Outcome at the Time of Last Observation

The outcome at the time of last observation will be classified as: recovered/resolved; recovered/resolved with sequelae; recovering/resolving; not recovered/not resolved; fatal; or unknown.

### Reporting of Serious Adverse Events

Initial and follow-up SAE reports must be completed by the investigator or designee and sent to the contract research organization (CRO) within 24 hours of the first awareness of a SAE. The investigator or designee must complete, sign and date the appropriate SAE form and verify the accuracy of the information against corresponding source documents. This information is to be sent to the CRO Pharmacovigilance Department.

### Serious Adverse Event Definition

An SAE is any untoward medical occurrence, whether considered to be related to investigational product or not, that at any dose: results in death; is life-threatening; requires inpatient hospitalization or prolongation of existing hospitalization; results in persistent or significant disability/incapacity; is a congenital anomaly; or is an important medical event.

Note that the term “life-threatening” in the definition of “serious” refers to an event in which the subject was at risk of death at the time of the event; it does not refer to an event which hypothetically might have caused death if it were more severe.

Note that inpatient hospitalization is defined as 24 hours in a hospital or an overnight stay. An elective hospital admission to treat a condition present before exposure to the test drug, or a hospital admission for a diagnostic evaluation of an AE, does not qualify the condition or event as an SAE. Further, an overnight stay in the hospital that is only due to transportation, organization, or accommodation problems and without medical background does not need to be considered an SAE.

Note that a congenital anomaly in an infant born to a mother who was exposed to the investigational product during pregnancy is an SAE. However, a newly diagnosed pregnancy in a subject that has received an investigational product is not considered an SAE unless it is suspected that the investigational product interacted with a contraceptive method and led to the pregnancy.

Note that medical and scientific judgment should be exercised in deciding whether it is appropriate to consider other situations serious, such as important medical events that may not be immediately life-threatening or result in death or hospitalization but may jeopardize the subject or may require intervention to prevent one of the other outcomes listed in the definition above. Examples of such events are intensive treatment in an emergency room or at home for allergic bronchospasm, blood dyscrasias or convulsions that do not result in hospitalization, or development of drug dependency or drug abuse.

### Serious Adverse Event Collection Time Frame

All SAEs, regardless of the relationship to study, are collected from the time the subject signs the informed consent until the subject's last visit (office or telephone contact). The investigator or designee must report all SAEs promptly to the CRO within 24 hours of first becoming aware of the event.

Any SAE(s), regardless of relationship to study drug, that occurred during the study, but which is not discovered by the study site until after study has completed must be reported to the CRO within 24 hours of the first awareness of the event.

### Serious Adverse Event Onset and Resolution Dates

The onset date of the SAE is defined as the date the event meets serious criteria. An AE which begins as a non-serious event, which later meets the definition of an SAE, should be entered once for the non-serious portion of the AE, and then be re-recorded as a new event with the start date the day it became serious.

The resolution date is the date an outcome is reached, stabilization is achieved (ie, the investigator does not expect any further improvement or worsening of the event), or the event is otherwise explained.

### Fatal Outcome

Fatal should only be designated as an outcome when the AE results in death. If more than 1 AE is possibly related to the subject's death, the outcome of death should be indicated for each such AE.

Any AE that results in the subject's death must have fatal checked as an outcome with the date of death recorded as the resolution date. AEs resulting in death must be reported within 24 hours as a SAE, if not already reported as such.

For other AEs, ongoing at the time of death that did not contribute to the subject's death, the outcome should be considered not resolved, without a resolution date recorded.

### Pregnancy

All females of childbearing potential, and males with female partners of childbearing potential, who participate in the study should be counseled on the need to utilize a highly effective method of birth control throughout the study and for 28 days following the last dose of study drug, and on the importance of avoiding pregnancy during study participation. A highly effective method of birth control is defined as one that results in a low failure rate (ie, <1% per year) when used consistently and correctly, such as oral/injectable/inserted/implanted/transdermal contraceptives, condom with diaphragm, condom with spermicide, diaphragm with spermicide, intrauterine hormone-releasing system or intrauterine device (IUD), or sexual abstinence. Contraception is not required where at least 6 weeks have passed since sterilization, defined as females having undergone one of the following surgeries: hysterectomy, bilateral tubal ligation or occlusion, bilateral oophorectomy, or bilateral salpingectomy; and males who are vasectomized. Contraception is not required where females are postmenopausal (12 consecutive months of spontaneous amenorrhea and age ≥51 years).

Females and males with female partners should be instructed to contact the investigator or study staff immediately if pregnancy occurs or is suspected.

Pregnancy testing will be conducted on every female as per the schedule of assessments (Table 1). A female who is found to be pregnant at Screening will be excluded from the study and considered to be a screening failure. A female who is found to be pregnant after receiving investigational product is required to be discontinued from the study and the end of study visit assessments performed as soon as possible after learning of the pregnancy.

The investigator must report the pregnancy of any female (study participant or female partner of male study participant) who becomes pregnant during investigational product treatment or within 28 days of discontinuing the investigational product (permission must be obtained from the pregnant female partner of a male patient to follow the pregnancy to conclusion and report the results). The pregnancy must be reported within 24 hours of learning of the pregnancy to the CRO using the Pregnancy Data Collection Form via the same fax and email address as for SAE reporting. The investigator should contact the designated individual(s) who receive pregnancy notification and record information related to the pregnancy on the Pregnancy Form/other designated form provided by the Sponsor or its designee.

The investigator is also responsible for following the pregnancy until delivery or termination. These findings must be reported on the Pregnancy Data Collection Form and forwarded to the designated individual(s). The event meets the SAE criterion only if it results in a spontaneous abortion or a congenital anomaly.

### Reporting to Regulatory Agency, Institutional Review Board/Ethics Committee and Site

The Sponsor or its designee is responsible for notifying the relevant regulatory authorities and if applicable, US central institutional review board (IRB) of related, unexpected SAEs.

In addition, the Sponsor or its designee is responsible for notifying active sites of all related, unexpected SAEs occurring during all interventional studies across the development program.

The investigator is responsible for notifying the local IRB, local ethics committee (EC), or the relevant local regulatory authority of all SAEs that occur at his/her site, as required.

### Example 1.6—Overdose and Medication Error

Overdose or medication error of investigational product, defined below (Table 6), must be reported to the Sponsor using the SAE reporting procedures outlined above whether or not they result in an AE/SAE. The 24-hour reporting period for SAEs does not apply to overdose or medication error event(s) unless the overdose or medication error event results in an SAE.

TABLE 6
Definition of Overdose and Medication Error

| Category | Definition |
|---|---|
| Overdose | Intentional or unintentional intake of a dose of |
| | investigational product exceeding the dose |
| | prescribed to the subject as part of the clinical trial. |
| Medication | Error made in prescribing, dispensing, administration |
| Error | and/or use of investigational product. Medication |
| | errors are reportable to the Sponsor or its |
| | designee if it involves: |
| | Dispensing, administration, and/or use of an |
| | unassigned treatment (for example, incorrect |
| | investigational product kit used by subject). |
| | Dispensing, administration and/or use of |
| | expired investigational product. |

Missing doses are not considered a medication error event and do not need reporting. Note that an overdose or medication error event can meet one or both of the above categories.

### Example 1.7—Safety Review Committee

An SRC will be involved in the conduct of this study. The SRC will be comprised of representatives of the sponsor and the study sites. The SRC has the responsibility for monitoring the clinical study's progress and the safety of the participating subjects.

After enrollment of 5 subjects at the initial level of 1 mg/day (0.5 mg twice daily), the SRC will convene to review cumulative safety data and any available PK and PD data, considering any treatment-emergent Grade 3 or higher AEs in the first dosing cohort, and make a recommendation if proceeding to the next dose level of 2 mg/day (1 mg twice daily) is warranted. The SRC may also suggest an alternate dosing level based on the safety, PK or PD data.

The SRC may meet on an ad-hoc basis to review reported AEs during the course of the study, including in the event of a treatment emergent SAE, if a safety signal is detected, or if stopping rules are met.

Upon completion of study enrollment, the SRC will convene to review all available safety, PK and PD data for the study population and document any recommendations for further safety monitoring and dosing of AVTX-006 in this patient population.

### Example 1.8—Statistics

Approximately 10 subjects are planned to be enrolled. AVTX-006 will be investigated in subjects with lymphatic malformations for the first time in this study. Hence, the sample size is based on feasibility and not on hypothesis testing.

This study will have the following populations of interest: The Enrolled Population includes all subjects who are enrolled; and the Safety Population includes all subjects who are enrolled and receive at least one treatment administration during this trial.

This section presents a summary of the planned statistical analyses. Additional statistical analyses, other than those described in this section may be performed if deemed appropriate. Details regarding data handling, analytical methods, and presentation of results will be provided in a Statistical Analysis Plan (SAP) for this study. The SAP will be finalized prior to database lock.

All safety, PK, PD, and efficacy variables will be summarized using descriptive statistics. Descriptive statistics for continuous data will include number of subjects (n), mean, standard deviation, median, minimum, and maximum. Summaries of change from baseline variables will include only subjects who have both a baseline value and corresponding value at the time point of interest. For all variables, baseline will be defined as the last assessment prior to the first dose of study drug. Descriptive statistics for categorical data will include frequency and percentage. All descriptive summaries will be produced by dose level and overall. The biomarker analysis will be detailed in the SAP. Detailed descriptions of data summaries and listings will be provided in the SAP.

The disposition of all subjects enrolled in this study will be summarized by dose level and completion/discontinuation status. Subjects who discontinue the study prematurely will be summarized by dose level and reason for discontinuation. The number of subjects in each analysis set will also be summarized by dose level.

All subject data will be reviewed for the occurrence of protocol deviations. Prior to database lock, all protocol deviations will be reviewed and classified with respect to the potential to influence experimental outcomes.

The analysis of demographic and baseline data will be performed for the Safety Population. Demographic variables include age, gender, race, ethnicity, height, weight, and body mass index. Demographics and other baseline characteristics will be summarized by dose level using descriptive statistics.

Medications will be coded using the World Health Organization Drug Dictionary. Prior and concomitant medications will be summarized by dose level.

Exposure to investigational product will be summarized by dose level using descriptive statistics. Subjects will be summarized according to cumulative exposure.

Treatment compliance will be measured using subject diaries. A subject will be considered non-compliant if their overall compliance is less than 80% or greater than 120%. This assessment will be completed at the end of the study.

Safety analyses will be conducted using data from the Safety Population. Safety variables include TEAEs, vital signs, slit lamp examination, and clinical laboratory results including renal function, ECG, ECHO, and imaging results. No formal inferential analyses will be conducted for safety variables, unless otherwise noted.

Adverse events will be coded using the Medical Dictionary for Regulatory Activities (MedDRA). An AE will be considered treatment-emergent if it occurs after the first dose of investigational product and through the Safety Follow-up visit (i.e., 7-10 days post a subject's last dose of investigational product).

The overall incidence of subjects having at least one AE will be summarized by dose level. The incidence of TEAEs will be summarized by dose level, system organ class (SOC), and preferred term (PT); each subject will be counted only once per SOC and PT. Similar summaries will be produced for SAEs and AEs leading to discontinuation. The maximum intensity of AEs and the maximum relationship to investigational product will also be summarized by dose level, SOC, and PT.

Clinical laboratory values will be compared to local laboratory normal ranges and flagged for levels of clinical concern. Summaries will focus on the frequencies of abnormal values as well as within-subject changes observed during the study.

Descriptive summaries for all reported values and change from baseline values will be summarized by laboratory test category, dose level, and visit.

Vital signs will be examined for changes during the study that may be attributed to exposure to investigational product.

Vital signs (systolic and diastolic blood pressure, pulse rate, respiratory rate, body weight, and body temperature) will be summarized by parameter, dose level, and visit using appropriate descriptive statistics.

Slit lamp exam results will be examined for changes during the study that may be attributed to exposure to investigational product. Slit lamp exam results will be summarized by dose level, and visit using appropriate descriptive statistics.
All PK parameters will be summarized using descriptive statistics by dose level and visit. Descriptive statistics for serum concentrations will include n, number of subjects with concentrations below the level of quantification (BLQ), mean, standard deviation, coefficient of variation, median, minimum, and maximum. For descriptive summaries, serum concentrations reported as BLQ will be set to zero.

All PD parameters will be summarized by dose level and visit.

All efficacy measures will be summarized using descriptive statistics by dose level and visit. Clinical activity of AVTX-006 will be evaluated by quality-of-life assessment, pain assessment using a visual analog scale, Karnofsky Performance Status and Investigator assessment of disease. Radiologic assessment may be performed if clinically indicated.

No interim analysis is planned for this study.

Although the present invention has been described in some detail by way of illustration and example for purposes of clarity and understanding, the descriptions and examples should not be construed as limiting the scope of the invention. The disclosures of all patent and scientific literature cited herein are expressly incorporated herein in their entirety by reference.

1. A method of treating a complex lymphatic malformation (CLM), comprising administering to a human subject diagnosed with a CLM trans-4-[4-Amino-5-(7-methoxyl-1H-indol-2-yl)-imidazo[5,1-f][1,2,4]triazine-7-yl]-cyclohexanecarboxylic acid, or a pharmaceutically acceptable salt thereof, at a dose of 1 mg/day or 2 mg/day.

2. The method of claim 1, wherein the trans-4-[4-Amino-5-(7-methoxyl-1H-indol-2-yl)-imidazo[5,1-f][1,2,4]triazine-7-yl]-cyclohexanecarboxylic acid, or pharmaceutically acceptable salt thereof, is administered in a 28-day cycle.

3. The method of claim 1, wherein the dose is 0.5 mg administered twice daily.

4. The method of claim 1, wherein the dose is 1 mg administered twice daily.

5. The method of claim 1, wherein the 1 mg/day dose comprises a first 0.5 mg dose and a second 0.5 mg dose, wherein the second 0.5 mg dose is administered about 12 hours after the first mg dose.

6. The method of claim 1, wherein the 2 mg/day dose comprises a first 1 mg dose and a second 1 mg dose, wherein the second 1 mg dose is administered about 12 hours after the first 1 mg dose.

7. (canceled)

8. The method of claim 1, wherein the pharmaceutically acceptable salt is trans-4-[4-Amino-5-(7-methoxyl-1H-indol-2-yl)-imidazo[5,1-f][1,2,4]tri azine-7-yl]-cyclohexanecarboxylic acid, tromethamine salt.

9. The method of claim 1, wherein the complex lymphatic malformation is selected from generalized lymphatic anomaly, Gorham syndrome, kaposiform lymphangiomatosis, microcystic lymphatic malformation, capillary lymphatico/venous malformation, abnormalities of the central conducting lymphatic channels, venous lymphatic malformation, capillary lymphatic arterial venous malformation, and phosphatase tensin homolog (PTEN) hamartoma tumor syndrome/vascular lesions.

10. (canceled)

11. The method of claim 1, wherein the subject has at least one localized lesion that involves fluid accumulation in the scalp, face, neck, limbs, abdomen, and/or chest, and wherein the subject exhibits at least a 20% reduction in the size of the lesion from baseline to end of treatment as assessed using radiologic imaging.

12. (canceled)

13. (canceled)

14. The method of claim 11, wherein the subject achieves no evidence of disease as assessed using radiologic imaging.

15. (canceled)

16. The method of claim 1, wherein the lymphatic malformation is a cystic lymphatic malformation.

17. The method of claim 16, wherein the cystic lymphatic malformation is selected from macrocystic, microcystic, and mixed.

18. The method of claim 1, wherein the complex lymphatic malformation is classified as moderate to severe based on medical history, imaging, and/or disease-related complications requiring systemic control.

19. The method of claim 18, wherein the complication is selected from coagulopathy, chronic pain, recurrent cellulitis, ulceration, visceral and/or bone involvement, and cardiac dysfunction.

20. The method of claim 1, wherein the subject exhibits a reduction in pain as assessed on a Visual Analogue Scale (VAS).

21. The method of claim 1, wherein the subject exhibits an improvement in quality-of-life as assessed using 36-Item Short Form Health Survey (SF-36).

22. The method of claim 1, wherein the subject exhibits an improvement in Karnofsky Performance Status.

23. The method of claim 1, wherein the subject is not administered a treatment selected from sirolimus, mitogen-activated protein kinase (MEK) inhibitors, PIK3CA inhibitors, or interferon alfa 2b, vascular endothelial growth factor receptor 3 (VEGFR-3) inhibitors, erlotinib or other epidermal growth factor receptor inhibitor, a systemic steroid, other immunosuppressive agents and/or other systemic agents targeting lymphatic malformations at the same time as the trans-4-[4-Amino-5-(7-methoxyl-1H-indol-2-yl)-imidazo[5,1-f][1,2,4]triazine-7-yl]-cyclohexanecarboxylic acid, or pharmaceutically acceptable salt thereof.

24. (canceled)

25. (canceled)

26. The method of claim 1, wherein the subject is administered a topical steroid targeting lymphatic malformations at the same time as the trans-4-[4-Amino-5-(7-methoxyl-1H-indol-2-yl)-imidazo[5,1-f][1,2,4]triazine-7-yl]-cyclohexanecarboxylic acid, or pharmaceutically acceptable salt thereof.

27. (canceled)

28. (canceled)

29. (canceled)

30. The method of claim 1, wherein the subject is a pediatric subject.
